(19) United States
(12) Patent Application Publication
(10) Pub. No.: US 2026/0062446 A1
(43) Pub. Date: Mar. 5, 2026

(54) SCALABLE SYNTHESIS OF ANABAENOPEPTINS

(71) Applicant: WAYNE STATE UNIVERSITY, DETROIT, MI (US)

(72) Inventors: Jeremy Jacob KODANKO, Grosse Pointe Park, MI (US); Judy A. WESTRICK, Washington, MI (US); Megan Lee QUANDT, Detroit, MI (US)

(73) Assignee: WAYNE STATE UNIVERSITY, DETROIT, MI (US)

(21) Appl. No.: 19/311,628

(22) Filed: Aug. 27, 2025

Related U.S. Application Data
(60) Provisional application No. 63/687,514, filed on Aug. 27, 2024.

(51) Int. Cl. C07K 7/56

(52) U.S. Cl. CPC C07K 7/56; C07B 2200/05

(57) ABSTRACT

A compound having formula I that is useful for preparing an anabaenopeptin or derivatives thereof is provided:

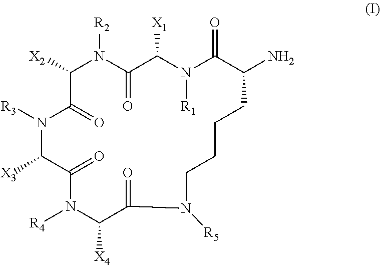

or a salt thereof and/or stereoisomers thereof and/or isotopic variations thereof, wherein: X₁, X₂, X₃, and X₄ are alkyl, aryl, hydroxyl, alkyl phenyl, alkyl phenol, and benzyl groups; and R₁, R₂, R₃, R₄, and R₅ are H or C₁₋₁₀ alkyl, wherein at least one of X₁, X₂, X₃, and X₄ is alkyl phenol, with the proviso that at least one of X₁, X₂, X₃, and X₄ are alkyl phenol.

### CROSS-REFERENCE TO RELATED APPLICATIONS

This application claims the benefit of U.S. provisional application Ser. No. 63/687,514 filed Aug. 27, 2024, the disclosure of which is hereby incorporated in its entirety by reference herein.

### STATEMENT REGARDING FEDERALLY SPONSORED RESEARCH OR DEVELOPMENT

This invention was made with Government support under Grant/Contract No. 1R01ES034017 awarded by the National Institutes of Health (NIH). The Government has certain rights in the invention.

### TECHNICAL FIELD

In at least one aspect, the present invention is related to the synthesis of anabaenopeptins and derivatives thereof.

### BACKGROUND

Harmful algal blooms (HABs), exacerbated by climate change and agricultural runoff—especially phosphorus and nitrogen—are becoming increasingly common in freshwater ecosystems. These blooms are predominantly composed of cyanobacteria, which produce toxic metabolites such as Anabaenopeptins (ABPs). The potential health impacts of these toxins are a growing concern, as HABs frequently occur in waterways used for recreational activities, exposing individuals and their pets to harmful toxins. However, the biological activity of these metabolites is not well understood, largely due to the difficulty in obtaining pure samples and the complexities involved in their detection. This knowledge gap presents significant public health risks.

Over 150 ABPs have been detected and isolated from freshwater and brackish cyanobacteria blooms. FIGS. 1A and 1B provide examples of ABPs. The ecological roles of ABPs are not well understood, but they are believed to serve as a defense mechanism against microbial parasites or to regulate cyanobacterial density. ABPs show significant potential to affect human health; many ABPs inhibit proteases and phosphatases at nanomolar concentrations, including chymotrypsin, trypsin, elastase, carboxypeptidases A and U (or B2), and protein phosphatases 1 and 2A. Studies have shown that ABPs can significantly impact aquatic ecosystems' ecology and food web structure, with intracellular concentrations exceeding 1000 μg/L in cyanobacterial blooms. ABPs also accumulate in fish that people consume and have been shown to harm nematodes and daphnia, indicating potential risks to aquatic and human health. Given their high concentrations in HABs and the key exposure pathways of ingestion or inhalation from aerosols, it is critical to understand the potential impact of ABPs on human health. High concentrations of ABPs are found in Planktothrix harmful algal blooms, with ingestion as a key exposure pathway, making it important to synthesize them to study their impact on the human gastrointestinal (GI) tract and microbiome.

ABPs may also become aerosolized, similar to microcystins (MCs), where lung exposure has the potential to be much more dangerous than ingestion. Furthermore, ABPs have been associated with toxic cyanobacterial blooms, where they were found at higher concentrations than other known toxins.

ABPs exhibit a wide range of biological activities, including potent inhibition of human enzymes such as carboxypeptidases A, B, and U (commonly called thrombin-activatable fibrinolysis inhibitor, TAFIa), highlighting their toxicological and pharmaceutical potential. Moreover, ABPs have been reported to inhibit protein phosphatases, raising concerns about their potential to impact basic cellular functions. Some ABPs, such as ABP F and Oscillamides B and C, have neurotoxic potential due to their ability to inhibit protein phosphatases, which are implicated in neurodegenerative diseases. Conversely, other ABPs serve as promising leads for developing new pharmaceuticals. ABPs have been recently described to inhibit TAFIa at low nanomolar concentrations, showing potential as antithrombin agents with a novel mechanism of action. Overall, ABPs represent a diverse group of bioactive peptides with significant ecological, toxicological, and pharmaceutical implications. Their structural complexity, biological activities, and interactions with other toxins underscore the importance of further research to fully understand their roles in aquatic environments and their impact on human health.

Unfortunately, only two ABP congeners, ABP A and B, are commercially available, and they are prohibitively costly (˜$3000/mg). The scarcity, high cost, and purity of ABP standards are major obstacles to HAB research and effective water and air management. The dearth of certified ABP standards is a major roadblock to research and responsible water and air management because the accuracy of results and validity of conclusions drawn from data calibrated to non-certified standards may be questionable or misleading. Without access to certified ABPs, biological studies to ascertain the potential risk or benefit of each compound to human health cannot be conducted. Without access to affordable research-grade ABP congeners, it is impossible to conduct biological studies to assess the potential health risks or benefits of each compound or to establish exposure routes. The primary cause of the scattered and unreliable availability of reference standards is that the existing approach is based on a natural product discovery and isolation workflow rather than a synthetic workflow, where ABPs are isolated from complex mixtures of many other cyanopeptides, including MCs. Challenges in isolating pure ABPs from these complex mixtures raise the risk that trace contaminants are actually the cause of some observed biological activity rather than specific ABPs under study. For example, MCs inhibit PP1 and IIa phosphatases at ng/mL levels, so there is a significant risk that much of the literature data for inhibition by ABPs is actually due to trace MC contaminants.

Genes involved in ABP biosynthesis have been extensively studied in species such as Nostoc punctiforme, Sphaerospermopsis torques-reginae, Nodularia spumigena, and Planktothrix agardhii, which contain genes related to homophenylalanine biosynthesis and nonribosomal peptide synthetases. Unfortunately, cyanopeptide production by cyanobacteria is highly variable and depends significantly on specific environmental conditions and host genetics, making many congeners challenging to obtain. Efficient expression systems for ABPs have not yet been developed. Regarding the state of the field, some MCs can be expressed in E. coli, but there are limits to the number of MCs that can be produced. Given the potent biological activity of cyanopeptides, it is questionable whether efficient biological expression systems can be developed to produce cyanopeptides in sufficient quantities without harming the host organism. Although future advancements might make it possible to produce ABP biologically, it is crucial to take immediate action to establish a stable supply chain due to the rising occurrence of HABs driven by climate change.

HABs are a significant global concern due to their detrimental effects on the environment, human health, and economies. These blooms, characterized by the rapid proliferation of algae, can lead to the production of toxic cyanopeptides that pose numerous risks. The impacts of HABs extend to marine life, fisheries, tourism, and public health. The detection of HABs is an active area of research, with a major goal to improve monitoring abilities. Climate change has also been identified as a contributing factor to the increase in HAB occurrences, emphasizing the importance of understanding the interactions between environmental changes and algal communities. Addressing the challenges posed by HABs requires interdisciplinary research efforts to enhance our understanding of their causes, impacts, and potential mitigation strategies.

Accordingly, there is a need for improved methodology for studying and synthesizing anabaenopeptins.

### SUMMARY

In at least one aspect, a compound having formula I that is useful for preparing an anabaenopeptin or derivatives thereof is provided:

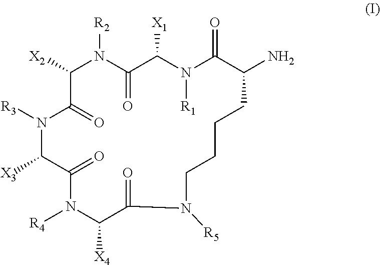

or a salt thereof and/or stereoisomers thereof and/or isotopic variations thereof,
wherein:
X₁, X₂, X₃, and X₄ are alkyl, aryl, hydroxyl, alkyl phenyl, alkyl phenol, and benzyl groups; and
R₁, R₂, R₃, R₄, and R₅ are H or C₁₋₁₀ alkyl, wherein at least one of X₁, X₂, X₃, and X₄ is alkyl phenol, with the proviso that at least one of X₁, X₂, X₃, and X₄ are alkyl phenol.

In another aspect, a method for making the compounds set forth above includes a step of preparing a linear pentapeptide that includes residues of amino acids having the following formula:

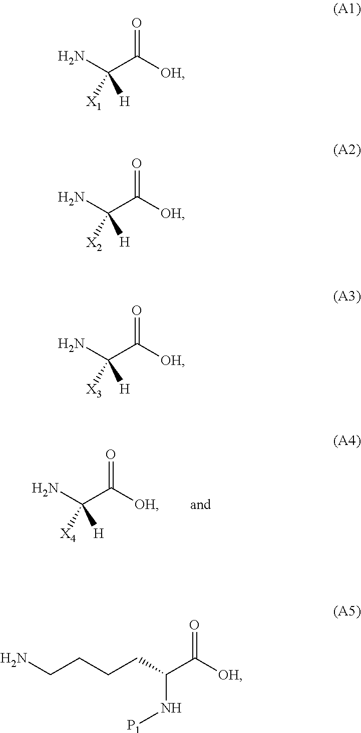

with the proviso that at least one of X₁, X₂, X₃, and X₄ are alkyl phenol. The pentapeptide is cyclized to form the compound having formula (I).

In another aspect, a scalable synthesis route to address the need for access to Anabaenopeptins (ABPs) for environmental monitoring is provided. This synthesis method incorporates the costly homotyrosine at a later stage, enabling the production of a common core pentacycle on a gram scale. This approach facilitates the generation of a diverse array of ABP derivatives through urea formation, providing a more efficient and accessible means of producing ABPs for research and monitoring purposes.

In another aspect, over 100 mg quantities of specific ABPs, such as ABPs C, D, J, 679, and Ferintoic Acid A are produced from a common pentacyclic core. The inventors aim to develop “certified” analytical reference standards for these ABPs and characterize their inhibitory properties on carboxypeptidases A and B and protein phosphatases I and IIA using these standards.

In another aspect, the use of hazardous chemicals like triphosgene can be eliminated and bypassed column purification bypassed in the initial steps, thereby conserving resources and reducing production risks. They have synthesized key ABPs, including Anabaenopeptin A, Anabaenopeptin D, Anabaenopeptin J, Anabaenopeptin 679, and Ferintoic Acid A, from a common pentacycle core. This approach enhances accessibility to ABPs for environmental monitoring and facilitates further research into their health impacts.

In another aspect, the invention focuses on characterizing Protein Phosphatase I (PPI) and Protein Phosphatase IIa (PP2A) inhibition are characterized using synthetic-derived ABP “certified” standards that do not contain MC contaminants. The inventors observed significant differences in IC50 values for CPA inhibition compared to literature values, indicating that synthetic-derived ABP 679 is over three orders of magnitude less potent than ABP A in CPA inhibition. This suggests that previous samples may have contained trace contaminants. The synthetic workflow eliminates contamination risks from other cyanopeptides, providing more reliable data and advancing the understanding of ABPs' impact on human health.

In another aspect, the inventors propose that de novo chemical synthesis is essential to bridge the gap until reliable biological expression systems are developed, if they ever are. Chemical synthesis is presented as an innovative and practical approach for producing ABPs and other cyanopeptides, offering a method that can be adopted immediately to meet current demands.

In another aspect, the inventors have developed an innovative synthetic approach that allows access to many ABP congeners from a single pentacyclic core. Unlike previous methods that required independent synthesis for each ABP derivative, this approach uses a common precursor and solution-phase synthesis, which is more scalable and cost-effective than solid-phase synthesis. This method provides a practical route for large-scale production of smaller peptides like ABPs, reducing overall costs and enhancing availability.

In another aspect, the invention addresses the high costs of ABPs, which are prohibitive for rigorous biological characterization. The inventors aim to disrupt the market by reducing ABP costs by over tenfold, making them affordable for researchers worldwide while maintaining strong profit margins. A recent cost analysis indicated that a gram of ABP A, currently valued at $3 million, can be produced from less than $5,000 in raw materials using their approach. This innovation aims to transform the ABP market, significantly enhancing monitoring and research capabilities to deepen the understanding of these important compounds.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIGS. 1A and 1B. Examples of anabaenopeptins.

FIGS. 2A, 2B, and 2C. Synthetic scheme for preparing a cyclic pentapeptide useful for preparing anabaenopeptins.

FIG. 3. Scheme for preparing a protected homotyrosine.

FIG. 4. Preparation of Anabaenopeptin 679 from the salt of the cyclic pentapeptide.

FIG. 5. Preparation of Anabaenopeptin A from the salt of the cyclic pentapeptide.

FIG. 6. Preparation of Anabaenopeptin A from the free base of the cyclic pentapeptide.

FIG. 7. Preparation of Anabaenopeptin D from the salt of the cyclic pentapeptide.

FIG. 8. Preparation of Anabaenopeptin D from the free base of the cyclic pentapeptide.

FIG. 9. Preparation of Anabaenopeptin J from the salt of the cyclic pentapeptide.

FIG. 10. Preparation of Anabaenopeptin J from the free base of the cyclic pentapeptide.

FIG. 11. Preparation of Ferintoic Acid A from the salt of the cyclic pentapeptide.

FIG. 12. Preparation of Ferintoic Acid A from the free base of the cyclic pentapeptide.

FIG. 13. Synthetic scheme for the experimental section.

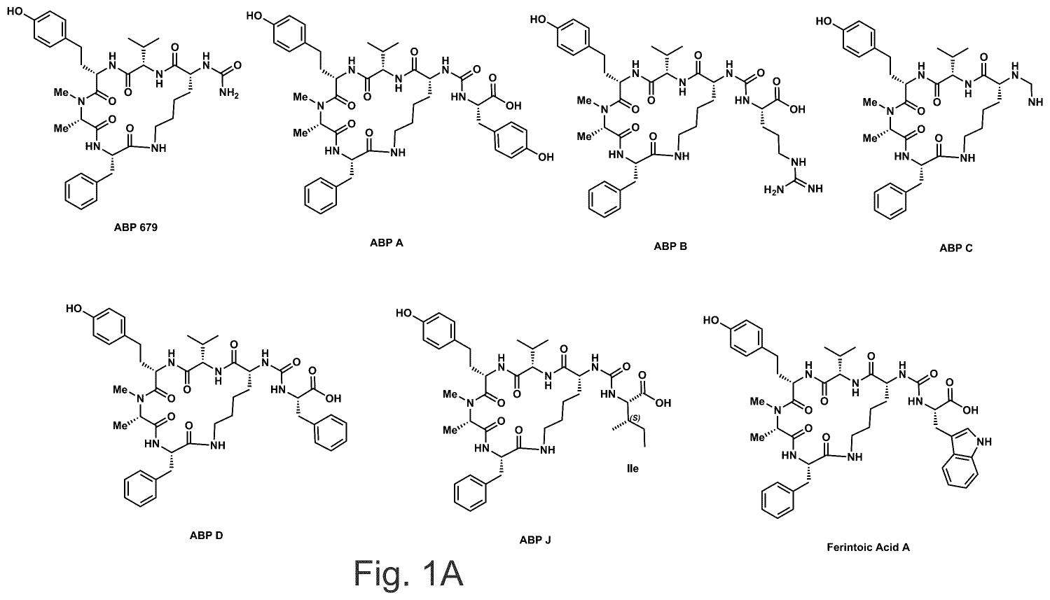

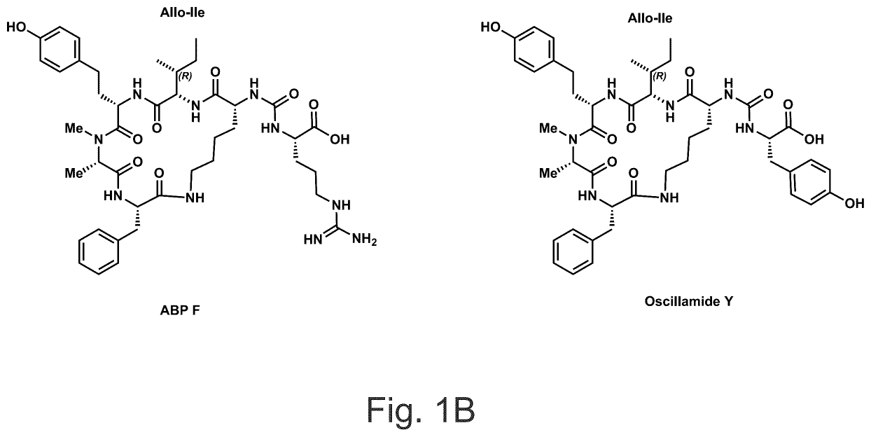

### DETAILED DESCRIPTION

Reference will now be made in detail to presently preferred compositions, embodiments, and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: all R groups (e.g. Rᵢ where i is an integer) include hydrogen, alkyl, lower alkyl, C₁₋₆ alkyl, C₆₋₁₀ aryl, C₆₋₁₀ heteroaryl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C₁₋₁₀ alkyl or C₆₋₁₈ aryl groups M is a metal atom (e.g., Na, K, Li, etc.) and L- is a counter anion (e.g., Cl—, Br—, tosylate, etc.); single letters (e.g., “n” or “o”) are 1, 2, 3, 4, or 5; in the compounds disclosed herein including compounds described by formula or by name, a CH bond can be substituted with alkyl, lower alkyl, C₁₋₆ alkyl, C₆₋₁₀ aryl, C₆₋₁₀ heteroaryl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C₁₋₁₀ alkyl or C₆₋₁₈ aryl groups M is a metal atom (e.g., Na, K, Li, etc.); percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

The term “alkyl” refers to C₁₋₂₀ inclusive, linear (i.e., “straight-chain”), branched, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl, or propyl, is attached to a linear alkyl chain. “Lower alkyl” refers to an alkyl group having 1 to about 10 carbon atoms (i.e., a C₁₋₈ alkyl), e.g., 1, 2, 3, 4, 5, 6, 7, or 8 carbon atoms. “Higher alkyl” refers to an alkyl group having about 11 to about 20 carbon atoms, e.g., 10, 11, 12, 13, 14, 15, 16, 17, 18, 19, or 20 carbon atoms. Compounds herein that reference alkyl can include lower or higher alkyl.

The term “amine” refers to primary amines, secondary amines, or tertiary amines. In a refinement, the amines have formula R¹NH₂ or R¹R²NH, or R¹R²R³N where R¹, R², and R³ are C₁₋₁₀ alkyl. In another refinement, amine refers to any organic compound with at least one CH bond.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

The phrase “composed of” means “including” or “comprising.” Typically, this phrase is used to denote that an object is formed from a material.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits. In the specific examples set forth herein, concentrations, temperature, and reaction conditions (e.g., pressure, pH, etc.) can be practiced with plus or minus 50 percent of the values indicated rounded to three significant figures. In a refinement, concentrations, temperature, and reaction conditions (e.g., pressure, pH, etc.) can be practiced with plus or minus 30 percent of the values indicated rounded to three significant figures of the value provided in the examples. In another refinement, concentrations, temperature, and reaction conditions (e.g., pH, etc.) can be practiced with plus or minus 10 percent of the values indicated rounded to three significant figures of the value provided in the examples.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

Throughout this application, where publications are referenced, the disclosures of these publications in their entirety are hereby incorporated by reference into this application to more fully describe the state of the art to which this invention pertains.

### Abbreviations:

“Alloc” means allyloxycarbonyl.

“BOC” means tert-butyloxycarbonyl.

“CBz” means carbobenzyloxy (also known as benzyloxycarbonyl)

Benzyl chloroformate

“Fmoc” means Fluorenylmethyloxycarbonyl

“Pht” means Phthalimide

“Tr” means Trityl.

In at least one aspect, a compound having formula I that is useful for preparing anabaenopeptin or derivative thereof is provided:

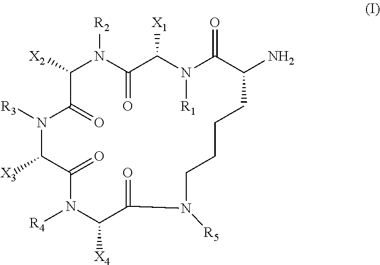

or a salt thereof and/or stereoisomers thereof and/or isotopic variations thereof,
wherein:
X₁, X₂, X₃, and X₄ are alkyl, aryl, hydroxyl, alkyl phenyl, alkyl phenol, and benzyl groups; and
R₁, R₂, R₃, R₄, and R₅ are H or C₁₋₁₀ alkyl, wherein at least one of X₁, X₂, X₃, and X₄ is alkyl phenol, with the proviso that at least one of X₁, X₂, X₃, and X₄ are alkyl phenol. In a refinement, at least two of X₁, X₂, X₃, and X₄ are alkyl phenol. In a refinement, the alkyl phenol is

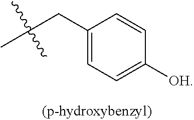 [SMILES: *Cc1ccc(O)cc1]

In another aspect, X₁ is isopropyl. In a further refinement, X2 and/or X3 is p-hydroxybenzyl. In still a further refinement, X₃ is methyl. In still a further refinement, X₄ is benzyl. In yet a further refinement, X₁ is isopropyl, X₂ is p-hydroxybenzyl, X₃ is methyl, and X₄ is benzyl. It should be appreciated that any combination of X₁ is isopropyl, X₂ is p-hydroxybenzyl, X₃ is methyl, and X₄ is benzyl is possible.

In another aspect, 1 to 3 of R₁, R₂, R₃, R₄, and R₅ are H, methyl, ethyl, propyl, isopropyl, butyl, isobutyl, sec-butyl, tert-butyl, pentyl, isopentyl, neopentyl, hexyl, or isohexyl.

In another aspect, at least one of X₁, X₂, X₃, and X₄ are not one of hydroxymethyl, hydroxyethyl, thiol, thioether, p-hydroxybenzyl, indolylmethyl, carboxymethyl, carboxyethyl, carboxamide, carboxyethylamide, butylammonium, guanidinium, imidazolylmethyl, or pyrrolidine.

In another aspect the compound having general formula I is a compound having specific formula II:

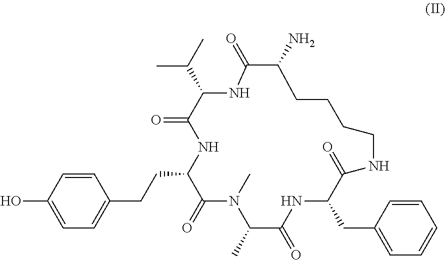

or a salt thereof and/or stereoisomers thereof and/or isotopic variations thereof.

In another aspect, the isotopic variations include, but are not limited to, hydrogen isotopes selected from the group consisting of protium (¹H), deuterium (²H), tritium (³H), and combinations thereof; and/or carbon isotopes selected from the group consisting of carbon-12 (¹²C), carbon-13 (¹³C), carbon-14 (¹⁴C), and combinations thereof; and/or oxygen isotopes selected from the group consisting of oxygen-16 (¹⁶O), oxygen-17 (¹⁷O), oxygen-18 (¹⁸O), and combinations thereof; and/or nitrogen isotopes selected from the group consisting of nitrogen-14 (¹⁴N), nitrogen-15 (¹⁵N), and combinations thereof.

In another aspect, a method for making the compounds set forth above includes a step of preparing a linear pentapeptide that includes residues of amino acids having the following formula:

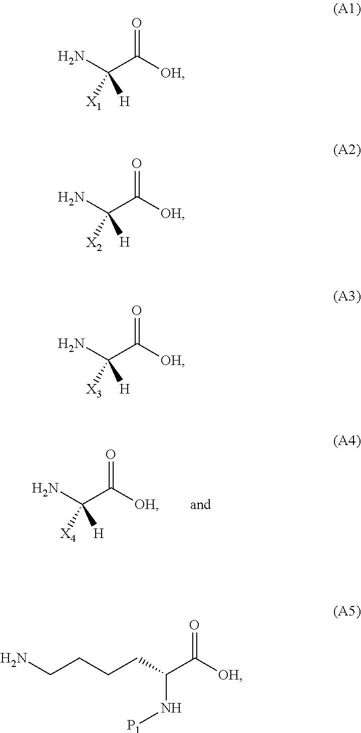

with the proviso that at least one of X₁, X₂, X₃, and X₄ are alkyl phenol. The pentapeptide is cyclized to form the compound having formula (I).

The linear pentapeptide is formed by the strategic application of protecting groups. Examples of protecting groups include BOC, Fmoc, Alloc, Cbz, and Tr. Boc is base stable and can be removed under acidic conditions (e.g., TFA). Fmoc is acid-stable and can be removed under basic conditions (e.g., piperidine). Alloc is acid and base-stable and can be removed by Pd(0) with a nucleophile. CBz is generally stable and can be removed via hydrogenation or with a strong acid. Pht is generally stable and can be removed with hydrazine. Tr is base stable and can be removed under acidic conditions (e.g., TFA).

In another aspect, to selectively add an amino acid to the C-terminus of the linear peptide, a strategy that ensures the protection of the N-terminus and any reactive side chains is applied. This approach involves protecting groups and activating the carboxyl group at the C-terminus for efficient peptide bond formation. The process begins by protecting the N-terminus of the peptide to prevent it from reacting during the coupling process. A commonly used protecting group for the N-terminus is Boc (tert-butoxycarbonyl), which is stable under basic conditions and can be easily removed with acid. By reacting a peptide with Boc, the N-terminus is transformed into a Boc-protected amine. This protection ensures that only the C-terminal carboxyl group is available for reaction. In addition to protecting the N-terminus, it may be necessary to protect the side chains of amino acids within the peptide, especially if they contain functional groups that could interfere with the coupling reaction. For instance, if the peptide contains amino acids with hydroxyl, thiol, or other nucleophilic groups, appropriate protecting groups like t-butyl for hydroxyl groups or trityl for thiol groups can be used to prevent unwanted side reactions. This step ensures that the C-terminus is the sole reactive site during coupling. Next, the carboxyl group at the C-terminus of the peptide can be activated to form a peptide bond with the incoming amino acid. Activation is typically achieved using coupling reagents such as DCC (dicyclohexylcarbodiimide), EDC (1-ethyl-3-(3-dimethylaminopropyl)carbodiimide), or HATU (O-(7-azabenzotriazol-1-yl)-N,N,N′,N′-tetramethyluroniumhexafluorophosphate). These reagents convert the carboxyl group into a more reactive species, such as an anhydride or active ester, facilitating the nucleophilic attack by the amine group of the incoming amino acid. To extend the C-terminus, the incoming amino acid must have its amino group protected to prevent it from reacting with other carboxyl groups. A common protecting group for the amino group of the incoming amino acid is the Fmoc (fluorenylmethyloxycarbonyl) or Boc, which is base-labile and can be removed under mild conditions. The incoming amino acid is thus used in its protected form. The coupling reaction proceeds with the activated peptide reacting with the Fmoc or Boc-protected amino acid, resulting in the formation of a new peptide bond and extending the peptide chain. This reaction selectively extends the C-terminus of the peptide, forming a covalent bond with the incoming amino acid. Following the coupling reaction, the protecting groups need to be removed to obtain the final unprotected peptide. The Boc group at the N-terminus can be removed using an acid like trifluoroacetic acid (TFA), yielding the free amine. Simultaneously, the Fmoc group on the added amino acid can be removed using a base such as piperidine, leaving the free amine group available for further reactions or analyses. Throughout this process, solvents like DMF (dimethylformamide) or DCM (dichloromethane) are used to optimize solubility and reaction conditions. Reaction progress can be monitored using techniques such as TLC (Thin Layer Chromatography) or HPLC (High-Performance Liquid Chromatography) to ensure successful coupling and deprotection. Upon completion, purification techniques like chromatography may be employed to achieve the desired purity of the extended peptide. By strategically using protecting groups and activating agents, selective addition of an amino acid to the C-terminus can be effectively controlled, allowing for precise peptide synthesis. This methodology is widely used in the synthesis of peptides and proteins, enabling the construction of complex biomolecules for research and therapeutic applications.

In another aspect, to selectively add an amino acid to the N-terminus of the linear peptide, a strategic approach involving protecting group chemistry is necessary to control the reaction site. This involves protecting the functional groups that are not intended to participate in the reaction, thus ensuring the selective extension of the N-terminus. First, the C-terminus of the peptide must be protected to prevent any reactions at this site during the coupling process. This can be achieved by converting the carboxyl group into an ester, such as a methyl ester, which masks its reactivity. In addition to protecting the C-terminus, it may be necessary to protect the side chains of amino acids within the peptide to prevent them from interfering with the coupling reaction. Protecting groups like Boc for amino side chains, t-butyl for hydroxyl groups, or trityl for thiol groups can be used depending on the specific amino acids present in the peptide. This step ensures that only the desired reactive site is exposed for the coupling reaction. Once the peptide is protected, the N-terminus needs to be activated to facilitate the coupling reaction. This can be achieved by converting the amino group into a reactive salt or by using a strong base to deprotonate it, thereby increasing its nucleophilicity. The activated peptide can then be coupled with an incoming amino acid that has its own carboxyl group activated. For example, a protected amino acid can be activated using coupling reagents like DCC (dicyclohexylcarbodiimide) or EDC (1-ethyl-3-(3-dimethylaminopropyl)carbodiimide) to form the activated ester. The activated ester reacts with the deprotonated amine at the N-terminus of the peptide, resulting in the formation of a new peptide bond and extending the peptide chain. After successful coupling, the protecting groups must be removed to obtain the final, unprotected peptide. The N-terminal protecting group, such as Cbz, can be removed through catalytic hydrogenation. Finally, the ester protecting the C-terminus is hydrolyzed using either base or acid, depending on the stability of the peptide and other functional groups, to yield the fully deprotected peptide. This strategy allows for precise control over the site of reaction, ensuring that the amino acid is added only to the N-terminus of the peptide. The choice of solvents such as DMF or DCM can aid in optimizing reaction conditions, and monitoring methods like TLC or HPLC ensure the reaction proceeds as planned. Upon completion, purification techniques such as chromatography may be employed to achieve the desired level of purity for the extended peptide. By carefully applying these techniques, peptide synthesis can be conducted with high selectivity and efficiency, which is crucial for applications in protein engineering and biochemistry.

In another aspect, the linear pentapeptide is selected form the group consisting of H₂N-A1-A2-A3-A4-A5-CO₂H; H₂N-A1-A2-A3-A5-A4-CO₂H; H₂N-A1-A2-A4-A3-A5-CO₂H; NH₂-A1-A2-A4-A5-A3-CO₂H; A1-A2-A5-A3-A4-CO₂H; NH₂-A1-A2-A5-A4-A3-CO₂H; NH₂-A1-A3-A2-A4-A5-CO₂H; NH₂-A1-A3-A2-A5-A4-CO₂H; NH₂-A1-A3-A4-A2-A5-CO₂H; NH₂-A1-A3-A4-A5-A2-CO₂H; NH₂-A1-A3-A5-A2-A4-CO₂H; NH₂-A1-A3-A5-A4-A2-CO₂H; NH₂-A1-A4-A2-A3-A5-CO₂H; NH₂-A1-A4-A2-A5-A3-CO₂H; NH₂-A1-A4-A3-A2-A5-CO₂H; NH₂-A1-A4-A3-A5-A2-CO₂H; NH₂-A1-A4-A5-A2-A3-CO₂H; NH₂-A1-A4-A5-A3-A2-CO₂H; NH₂-A1-A5-A2-A3-A4-CO₂H; NH₂-A1-A5-A2-A4-A3-CO₂H; NH₂-A1-A5-A3-A2-A4-CO₂H; NH₂-A1-A5-A3-A4-A2-CO₂H; NH₂-A1-A5-A4-A2-A3-CO₂H; NH₂-A1-A5-A4-A3-A2-CO₂H; NH₂-A2-A1-A3-A4-A5-CO₂H; NH₂-A2-A1-A3-A5-A4-CO₂H; NH₂-A2-A1-A4-A3-A5-CO₂H; NH₂-A2-A1-A4-A5-A3-CO₂H; NH₂-A2-A1-A5-A3-A4-CO₂H; NH₂-A2-A1-A5-A4-A3-CO₂H; NH₂-A2-A3-A1-A4-A5-CO₂H; NH₂-A2-A3-A1-A5-A4-CO₂H; NH₂-A2-A3-A4-A1-A5-CO₂H; NH₂-A2-A3-A4-A5-A1-CO₂H; NH₂-A2-A3-A5-A1-A4-CO₂H; NH₂-A2-A3-A5-A4-A1-CO₂H; NH₂-A2-A4-A1-A3-A5-CO₂H; NH₂-A2-A4-A1-A5-A3-CO₂H; NH₂-A2-A4-A3-A1-A5-CO₂H; NH₂-A2-A4-A3-A5-A1-CO₂H; NH₂-A2-A4-A5-A1-A3-CO₂H; NH₂-A2-A4-A5-A3-A1-CO₂H; NH₂-A2-A5-A1-A3-A4-CO₂H; NH₂-A2-A5-A1-A4-A3-CO₂H; NH₂-A2-A5-A3-A1-A4-CO₂H; NH₂-A2-A5-A3-A4-A1-CO₂H; NH₂-A2-A5-A4-A1-A3-CO₂H; NH₂-A2-A5-A4-A3-A1-CO₂H; NH₂-A3-A1-A2-A4-A5-CO₂H; NH₂-A3-A1-A2-A5-A4-CO₂H; NH₂-A3-A1-A4-A2-A5-CO₂H; NH₂-A3-A1-A4-A5-A2-CO₂H; NH₂-A3-A1-A5-A2-A4-CO₂H; NH₂-A3-A1-A5-A4-A2-CO₂H; NH₂-A3-A2-A1-A4-A5-CO₂H; NH₂-A3-A2-A1-A5-A4-CO₂H; NH₂-A3-A2-A4-A1-A5-CO₂H; NH₂-A3-A2-A4-A5-A1-CO₂H; NH₂-A3-A2-A5-A1-A4-CO₂H; NH₂-A3-A2-A5-A4-A1-CO₂H; NH₂-A3-A4-A1-A2-A5-CO₂H; NH₂-A3-A4-A1-A5-A2-CO₂H; NH₂-A3-A4-A2-A1-A5-CO₂H; NH₂-A3-A4-A2-A5-A1-CO₂H; NH₂-A3-A4-A5-A1-A2-CO₂H; NH₂-A3-A4-A5-A2-A1-CO₂H; NH₂-A3-A5-A1-A2-A4-CO₂H; NH₂-A3-A5-A1-A4-A2-CO₂H; NH₂-A3-A5-A2-A1-A4-CO₂H; NH₂-A3-A5-A2-A4-A1-CO₂H; NH₂-A3-A5-A4-A1-A2-CO₂H; NH₂-A3-A5-A4-A2-A1-CO₂H; NH₂-A4-A1-A2-A3-A5-CO₂H; NH₂-A4-A1-A2-A5-A3-CO₂H; NH₂-A4-A1-A3-A2-A5-CO₂H; NH₂-A4-A1-A3-A5-A2-CO₂H; NH₂-A4-A1-A5-A2-A3-CO₂H; NH₂-A4-A1-A5-A3-A2-CO₂H; NH₂-A4-A2-A1-A3-A5-CO₂H; NH₂-A4-A2-A1-A5-A3-CO₂H; NH₂-A4-A2-A3-A1-A5-CO₂H; NH₂-A4-A2-A3-A5-A1-CO₂H; NH₂-A4-A2-A5-A1-A3-CO₂H; NH₂-A4-A2-A5-A3-A1-CO₂H; NH₂-A4-A3-A1-A2-A5-CO₂H; NH₂-A4-A3-A1-A5-A2-CO₂H; NH₂-A4-A3-A2-A1-A5-CO₂H; NH₂-A4-A3-A2-A5-A1-CO₂H; NH₂-A4-A3-A5-A1-A2-CO₂H; NH₂-A4-A3-A5-A2-A1-CO₂H; NH₂-A4-A5-A1-A2-A3-CO₂H; NH₂-A4-A5-A1-A3-A2-CO₂H; NH₂-A4-A5-A2-A1-A3-CO₂H; NH₂-A4-A5-A2-A3-A1-CO₂H; NH₂-A4-A5-A3-A1-A2-CO₂H; NH₂-A4-A5-A3-A2-A1-CO₂H; NH₂-A5-A1-A2-A3-A4-CO₂H; NH₂-A5-A1-A2-A4-A3-CO₂H; NH₂-A5-A1-A3-A2-A4-CO₂H; NH₂-A5-A1-A3-A4-A2-CO₂H; NH₂-A5-A1-A4-A2-A3-CO₂H; NH₂-A5-A1-A4-A3-A2-CO₂H; NH₂-A5-A2-A1-A3-A4-CO₂H; NH₂-A5-A2-A1-A4-A3-CO₂H; NH₂-A5-A2-A3-A1-A4-CO₂H; NH₂-A5-A2-A3-A4-A1-CO₂H; NH₂-A5-A2-A4-A1-A3-CO₂H; NH₂-A5-A2-A4-A3-A1-CO₂H; NH₂-A5-A3-A1-A2-A4-CO₂H; NH₂-A5-A3-A1-A4-A2-CO₂H; NH₂-A5-A3-A2-A1-A4-CO₂H; NH₂-A5-A3-A2-A4-A1-CO₂H; NH₂-A5-A3-A4-A1-A2-CO₂H; NH₂-A5-A3-A4-A2-A1-CO₂H; NH₂-A5-A4-A1-A2-A3-CO₂H; NH₂-A5-A4-A1-A3-A2-CO₂H; NH₂-A5-A4-A2-A1-A3-CO₂H; NH₂-A5-A4-A2-A3-A1-CO₂H; NH₂-A5-A4-A3-A1-A2-CO₂H; and NH₂-5-A4-A3-A2-A1 where “-” between any 2 of A1, A2, A3, A4, and A5-represents a peptide bond.

In another aspect, a method for making the compounds set forth above includes a step a) of providing a first protected amino acid having formula P1 or a salt thereof and/or stereoisomers thereof and/or isotopic variations thereof:

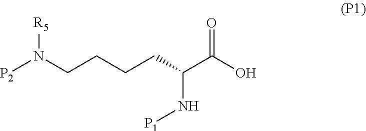

wherein P₁ is a first protecting group and P₂ is a second protecting group that can be selectively removed under different conditions. In step b), a second protected amino acid is attached to the C-terminus side of the first amino acid having formula 1 and then removing protecting group P₂ to form a peptide having formula P2 or a salt thereof and/or stereoisomers thereof and/or isotopic variations thereof with an exposed amino group:

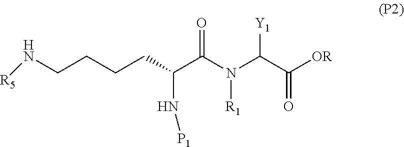

wherein Y₁ is X₁ or X₁ with a removable protecting group and OR is a C₁₋₆ alkoxyl protecting group. In step c), a third protected amino acid is attached to the exposed amino group to form a peptide having formula P3 or a salt thereof and/or stereoisomers thereof and/or isotopic variations thereof:

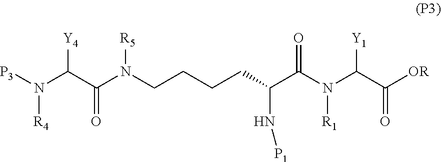

wherein Y₄ is X₄ or a X₄ with a removable protecting group and P₃ is a third protecting group. In step d), P₄ is removed and a fourth protected amino acid is attached to the N-terminus side of the peptide having formula P3 to form a peptide having formula P4 or a salt thereof and/or stereoisomers thereof and/or isotopic variations thereof:

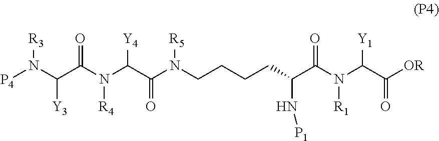

wherein Y₃ is X₃ or a X₃ with a removable protecting group and P₄ is a fourth protecting group. In step e), P₄ is removed and a fifth protected amino acid is attached to the N-terminus side of the peptide having formula 34 to form a peptide having formula 5 a salt thereof and/or stereoisomers thereof and/or isotopic variations thereof:

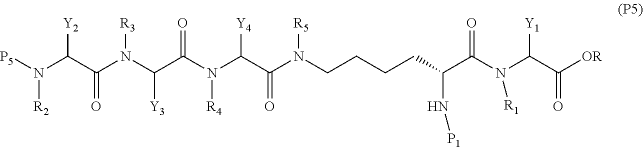

wherein Y₂ is X₂ or a X₂ with a removable protecting group and P₅ is a fifth protecting group. In step f), OR and P₅ are removed to form pentapeptide P6 or a salt thereof and/or stereoisomers thereof and/or isotopic variations thereof:

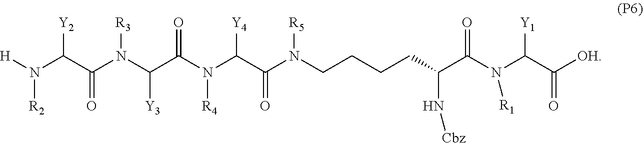

In step g), the peptide having formula P6 is cyclized to form pentacycle peptide having formula P7 or a salt thereof and/or stereoisomers thereof and/or isotopic variations thereof:

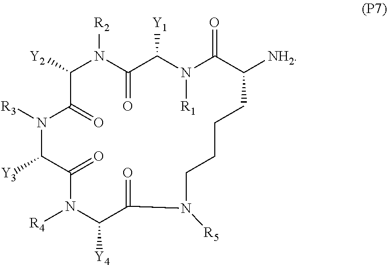

In another aspect, the method further includes a step of removing any protective groups from Y₁, Y₂, Y₃, and Y₄ to form:

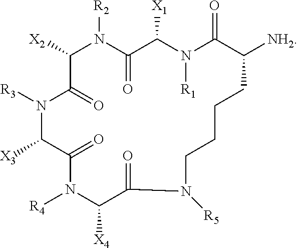

In another aspect, P₁ is removeable under different conditions that P₂, P₃, P₄, and P₅. In a refinement, P₁ is carbobenzyloxy and P₂, P₃, P₄, and P₅ are tert-butoxycarbonyl.

In another aspect, Y₁ is isopropyl. In a refinement, Y2 is a protected p-hydroxybenzyl. In a further refinement, Y₃ is methyl. In still a further refinement, Y₄ is benzyl. In yet a further refinement, Y₁ is isopropyl, Y₂ is p-hydroxybenzyl, Y₃ is methyl, and Y₄ is benzyl. It should be appreciated that any combination of Y₁ is isopropyl, X₂ is p-hydroxybenzyl, Y₃ is methyl, and Y₄ is benzyl is possible.

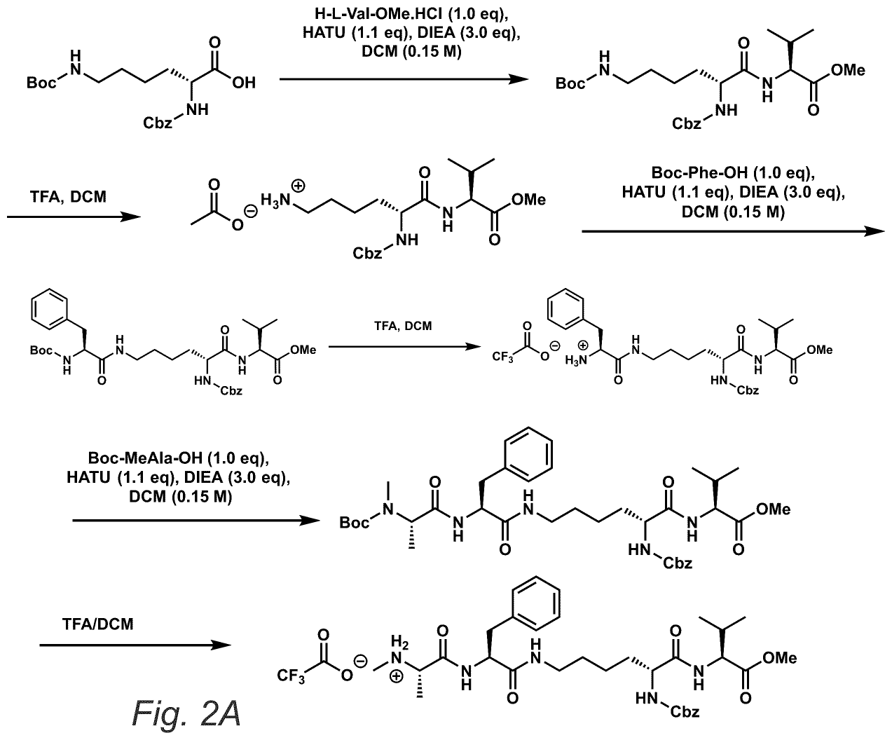

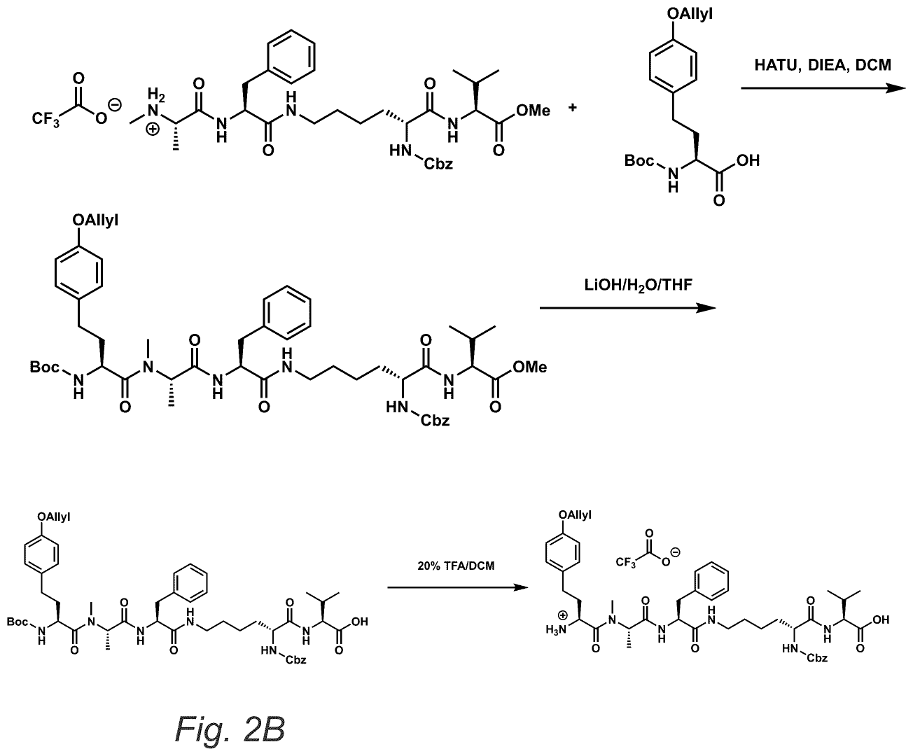

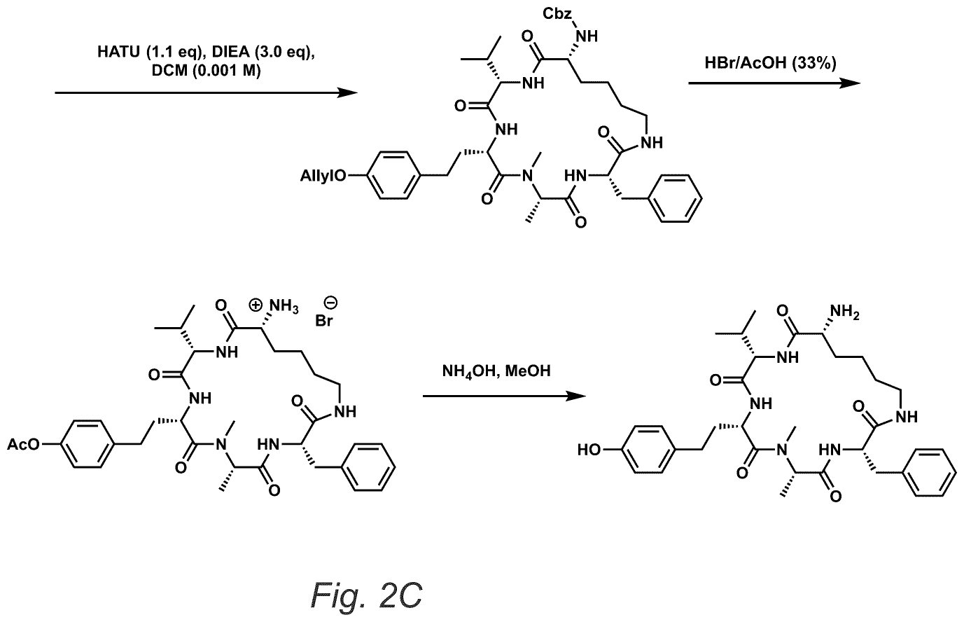

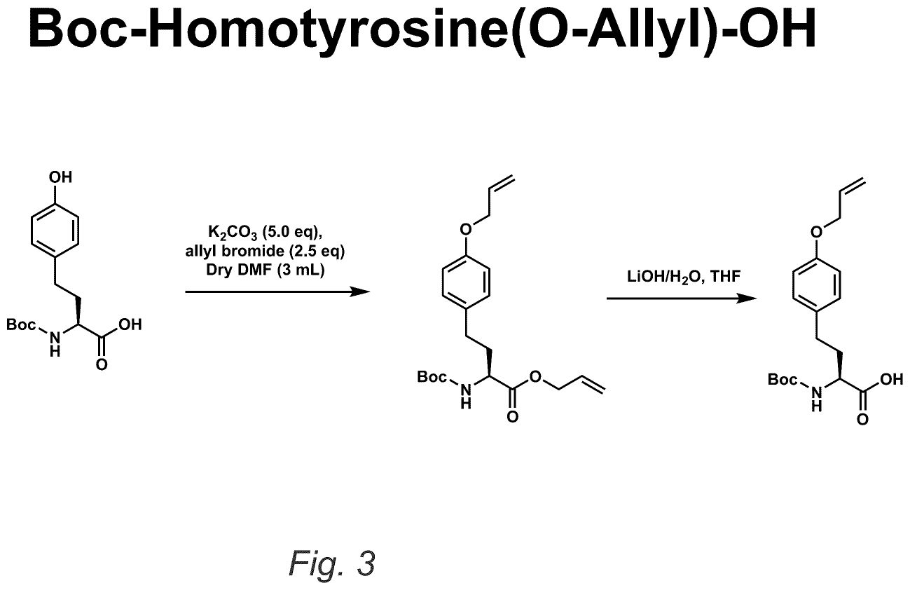

FIGS. 2A, 2B, and 2C provide a specific chemical scheme for a synthesis for a cyclic pentapeptide useful for preparing anabaenopeptins. FIG. 3 provides a scheme for preparing a protected homotyrosine.

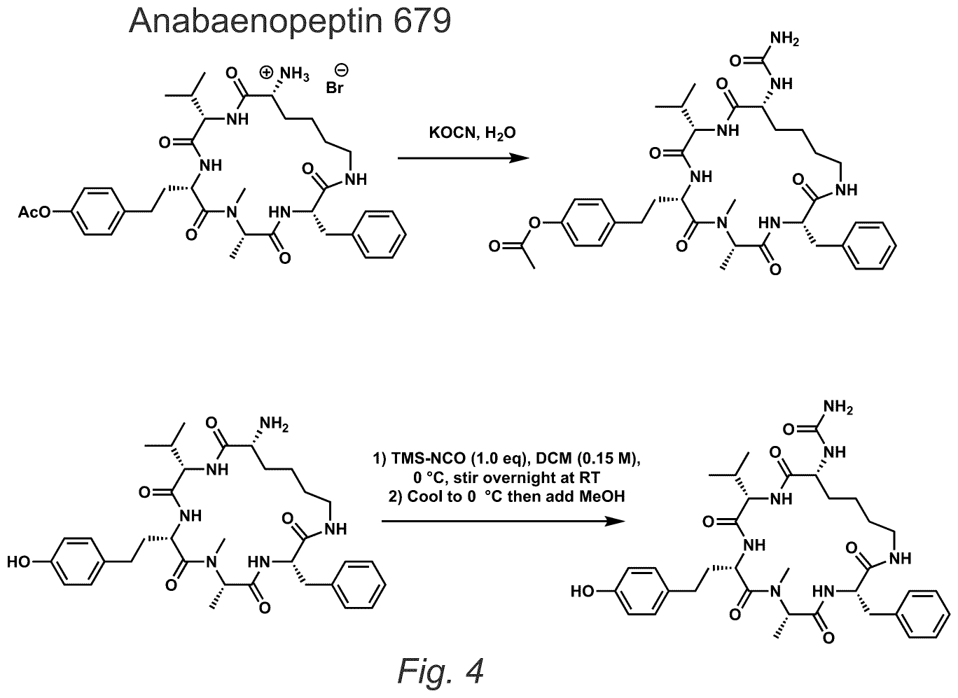

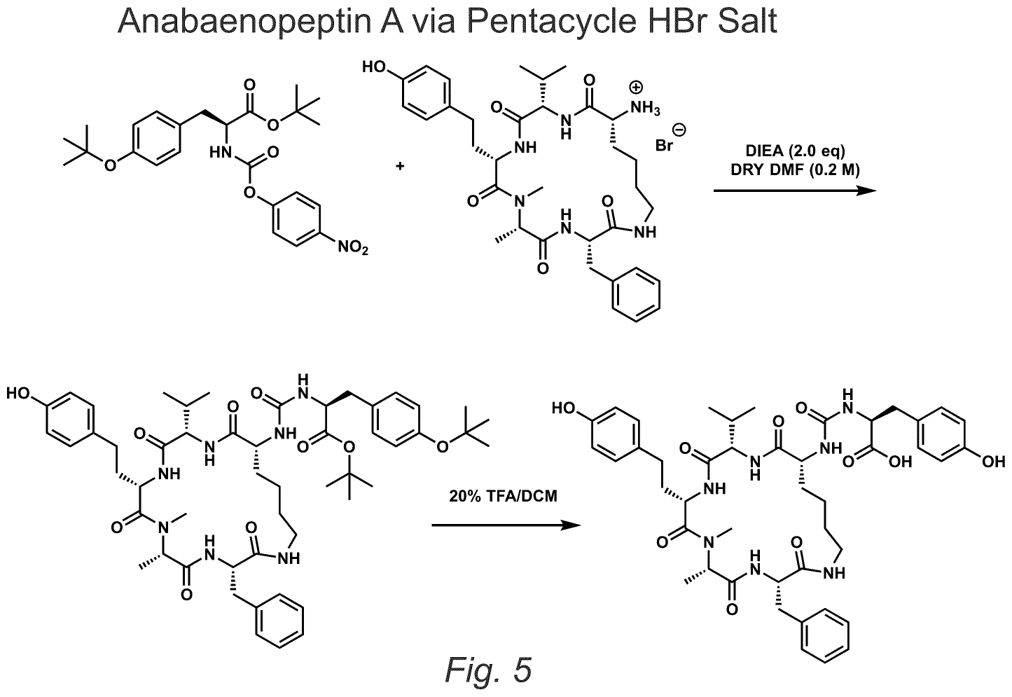

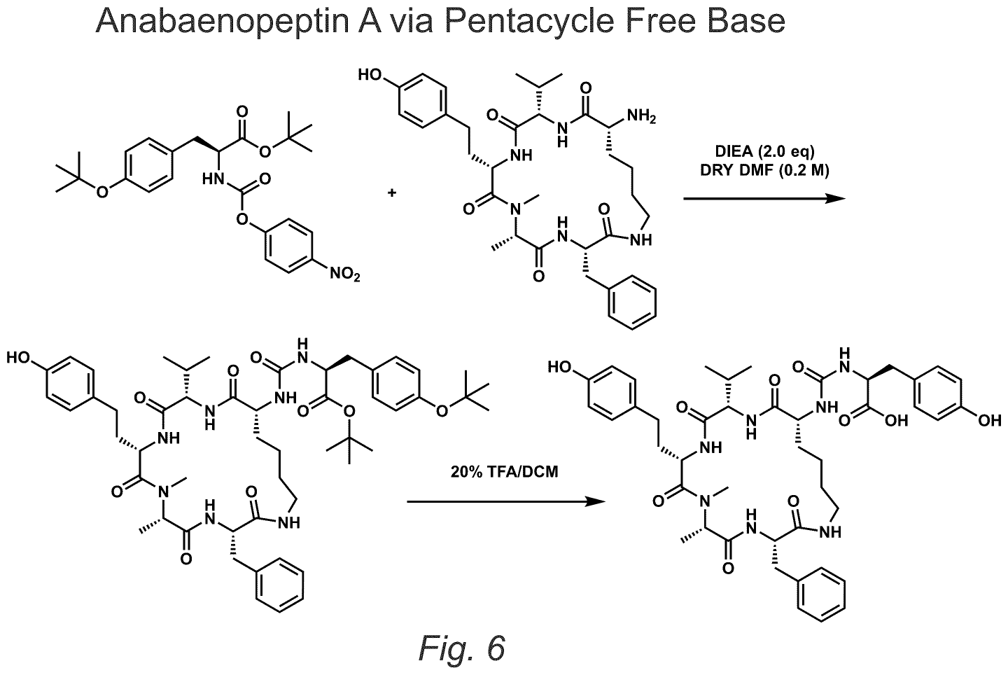

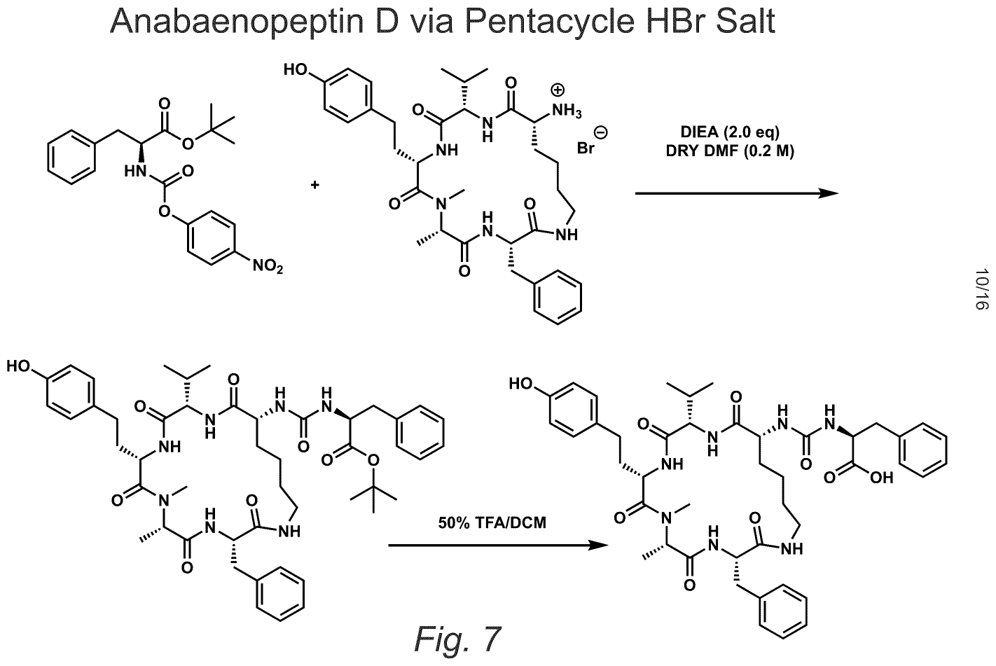

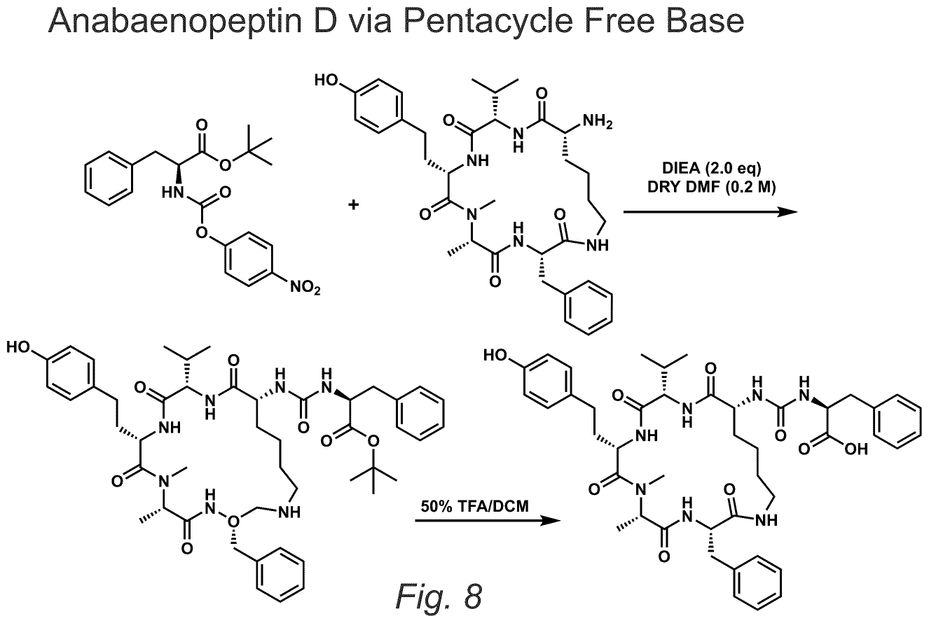

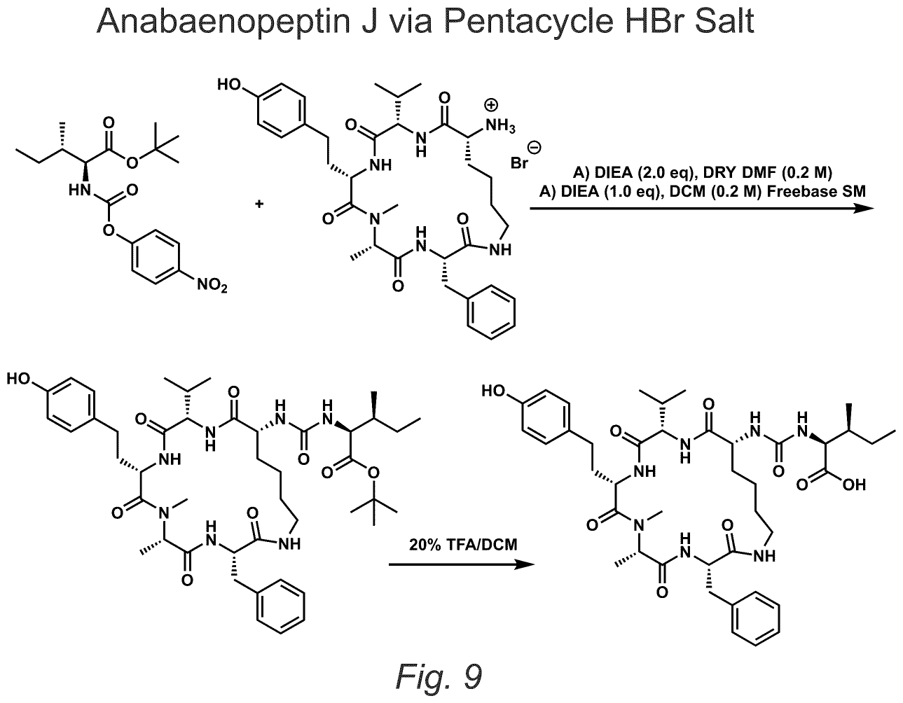

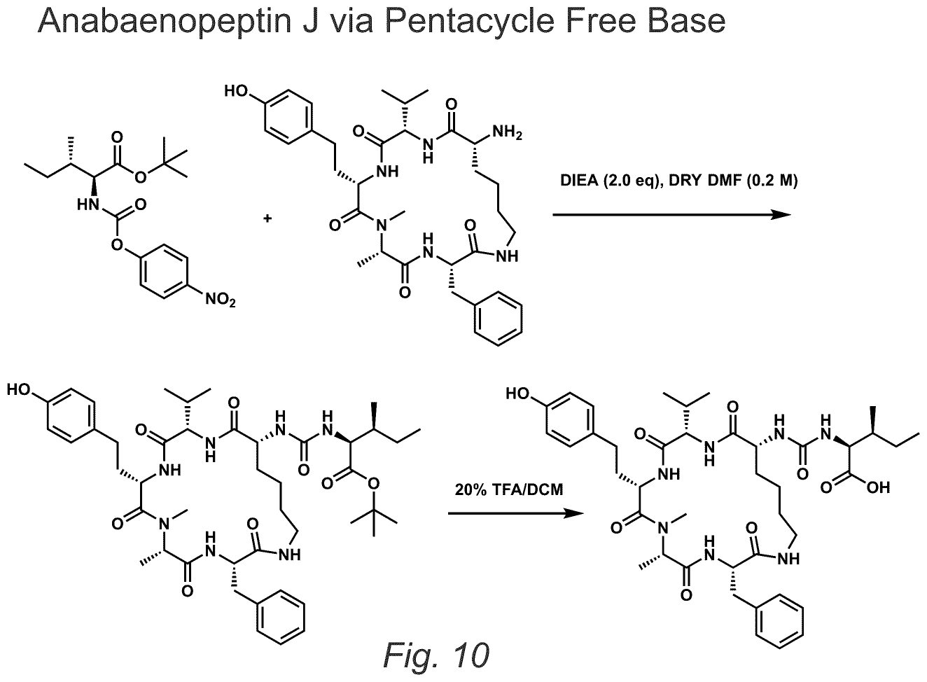

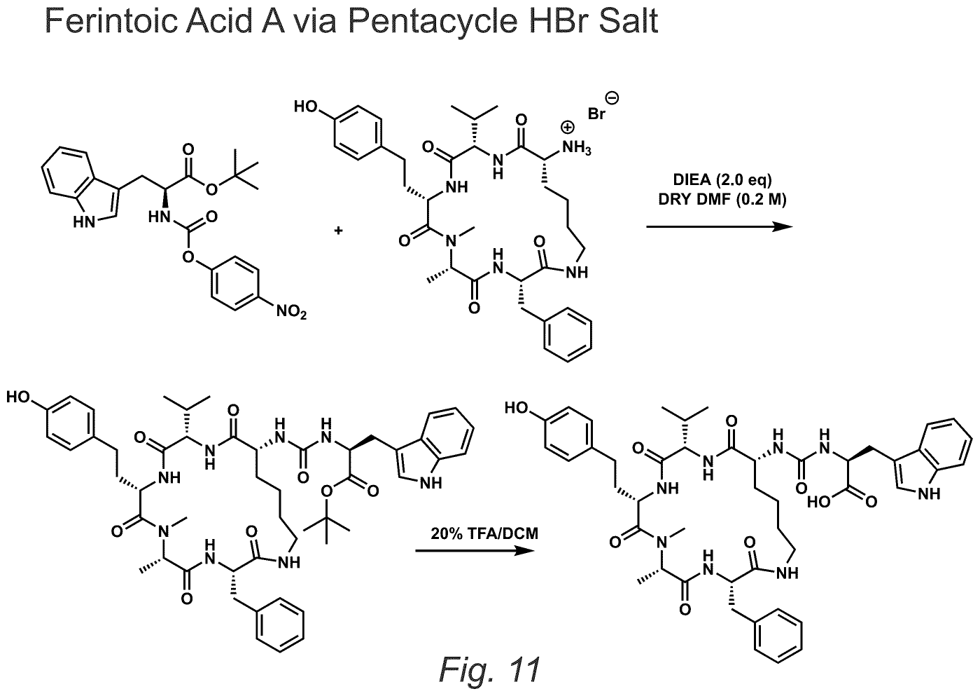

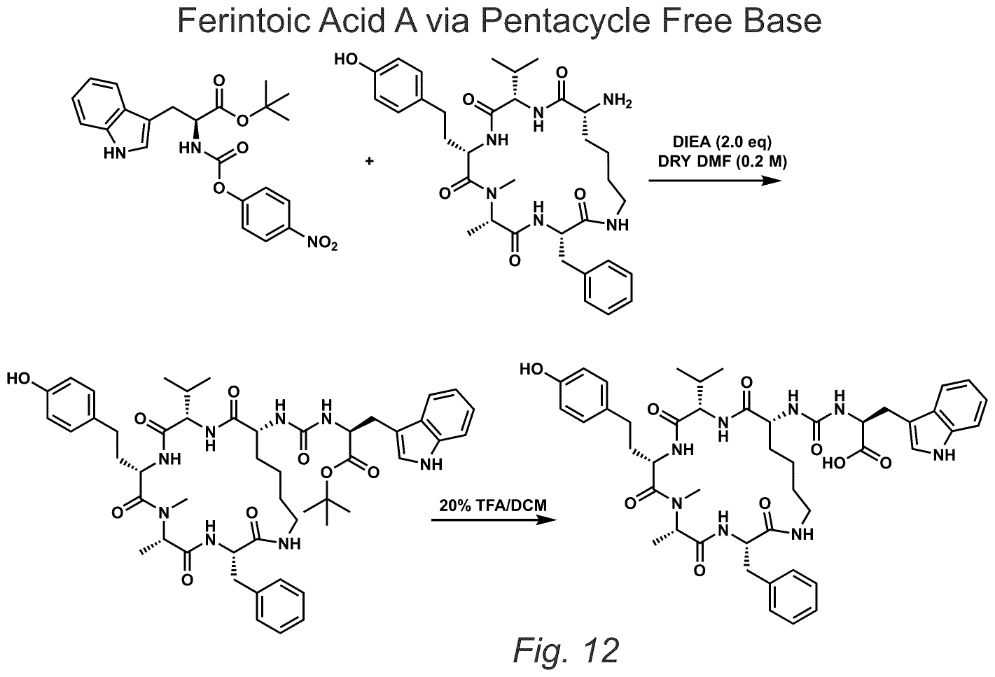

FIGS. 4-12 provide various synthetic schemes for making anabaenopeptin or derivative. In one example, a method for forming an anabaenopeptin or derivative thereof includes step a) of reacting the compounds set forth above based on formula I with a compound having formula III:

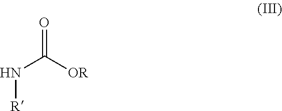

to form the anabaenopeptin or derivative thereof having formula IV:

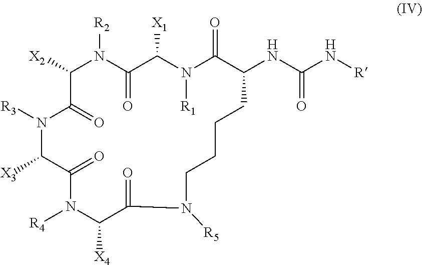

wherein:
R is C₁₋₁₀ alkyl, phenyl, or nitrophenyl; and
R′ is a C₄-20 hydrocarbon group that includes an ester moiety, a C₄₋₂₀ alkyl, C₆₋₂₀ aryl, or a C₇₋₂₀ alkyl aryl. In a refinement, the compound having formula III is selected from the group consisting of:

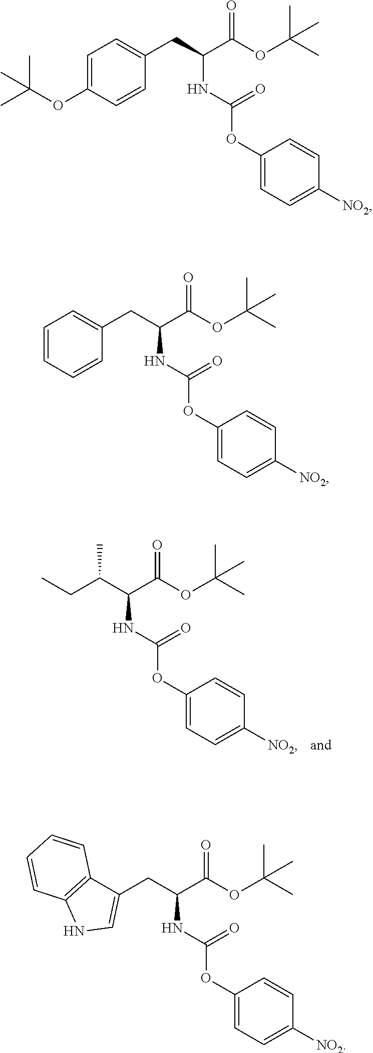 [SMILES: CC(C)(C)OC(=O)[C@H](Cc1ccc(OC(C)(C)C)cc1)NC(=O)Oc1ccc([N+](=O)[O-])cc1 ; CC(C)(C)OC(=O)[C@H](Cc1ccccc1)NC(=O)Oc1ccc([N+](=O)[O-])cc1 ; CC[C@H](C)[C@H](NC(=O)Oc1ccc([N+](=O)[O-])cc1)C(=O)OC(C)(C)C ; CC(C)(C)OC(=O)[C@H](Cc1c[nH]c2ccccc12)NC(=O)Oc1ccc([N+](=O)[O-])cc1]

In another example, a method for forming an anabaenopeptin or derivative thereof is provided. The method includes step a) of converting the compound having formula I into an isocyanate having formula V:

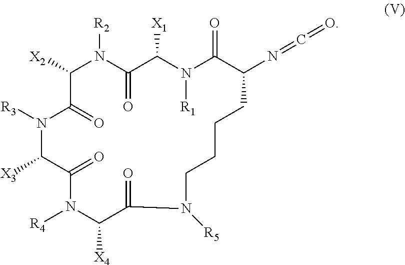

In a refinement, The method also includes a step of reacting the compound having formula V with an amine. In another refinement, the compound having formula I is reacted with triphosgene or triphosgene and a base to form the compound having formula V in step a). In a further refinement, the amine has formula VI:

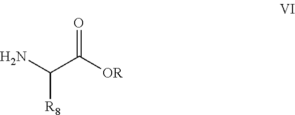

where R₈ is a C₄₋₂₀ hydrocarbon group that includes an ester moiety, a C₄₋₂₀ alkyl, C₆₋₂₀ aryl, or a C₇₋₂₀ alkyl aryl. A specific example of such an amine is:

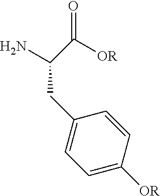

where R is H or C1-10 alkyl.

In another example, a method for forming an anabaenopeptin or derivative thereof is provided. The method includes a step of reacting the compound set forth above with a compound having formula III

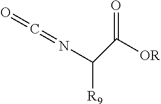

to form the anabaenopeptin or derivative thereof having formula VII:

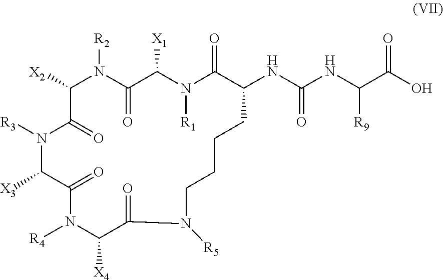

wherein:
R₉ is a C₄₋₂₀ hydrocarbon group, a C₄₋₂₀ alkyl, C₆₋₂₀ aryl, or a C₇₋₂₀ alkyl aryl; and
R′ is a C₄₋₂₀ hydrocarbon group that includes an ester moiety, a C₄₋₂₀ alkyl, C₆₋₂₀ aryl, or a C₇₋₂₀ alkyl aryl.

The following examples illustrate the various embodiments of the present invention. Those skilled in the art will recognize many variations that are within the spirit of the present invention and scope of the claim

### General Information

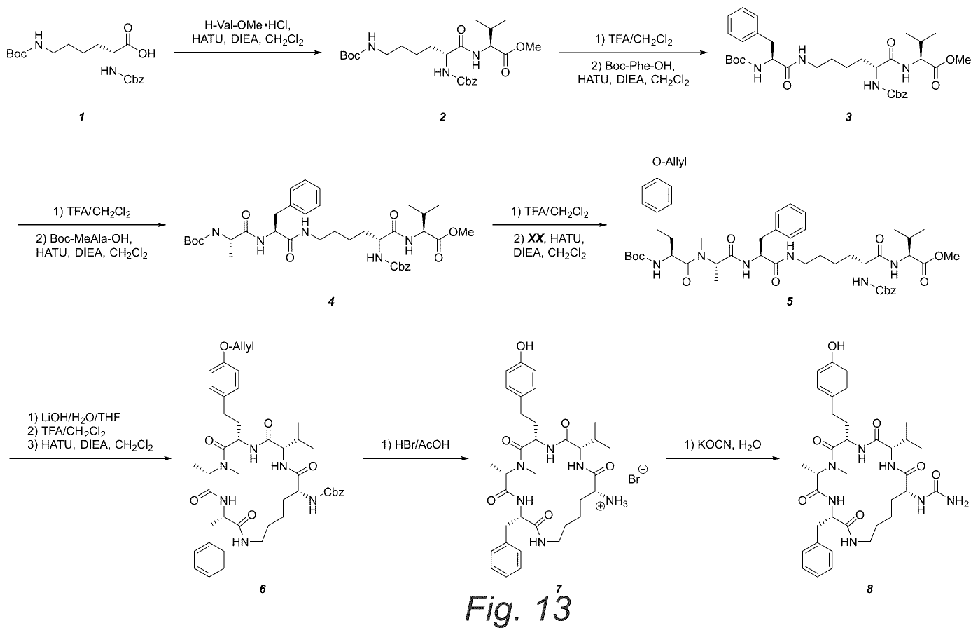

FIG. 13 provides a Synthetic scheme for this section. Unless stated otherwise, all commercially available reagents were used as received without additional purification. ¹H NMR and ¹³C NMR spectra were recorded on either a Varian Mercury-400 MHz or a Bruker BioSpin GmbH-500 MHz spectrometer. High-resolution mass spectral analyses were performed by the Lumigen Instrument Center, Wayne State University, using the Thermo LTQ-Orbitrap ESi-Positive, supported by R01 GM098285-07S1. Purifications were carried out using silica gel 230-400 Mesh, Grade 60 Å. All reported yields correspond to chromatographically and spectroscopically pure products. IR data was obtained on a Bruker Tensor27 FT-infrared spectrometer. Optical rotation data were collected on a Jasco P-2000 Polarimeter equipped with a tungsten-halogen lamp and measured with a 589 nm filter at the specified concentration. Enzyme inhibition assays utilized a Tecan Spark plate reader for absorption data collection.

General Procedure for Peptide Coupling. A solution of carboxylic acid (1.0 eq) in dichloromethane (CH₂Cl₂) (0.15 M) was treated with hexafluorophosphate azabenzotriazole tetramethyl uronium (HATU) (1.1 equiv) followed by N,N-diisopropylethylamine (DIEA) (2.0-3.0 equiv). After maintaining the solution for 10 min at room temperature (25° C.), the amine (1.0 eq) was added in one portion. Reaction mixtures were monitored by TLC for consumption of starting materials, then concentrated in vacuo. Crude reaction mixtures were dissolved in ethyl acetate (EtOAc) and the organic layer was washed with aqueous solutions of ammonium chloride (NH₄Cl), sodium bicarbonate (NaHCO₃), and sodium chloride. The organic layer was dried over sodium sulfate (Na₂SO₄), filtered, and concentrated in vacuo. The crude products were purified by recrystallization or by flash column chromatography as described below.

General Procedure for Boc Deprotection. Solutions of Boc-protected peptides (1.0 eq) were treated with 20% TFA in CH₂Cl₂ at room temperature and were monitored by TLC for consumption of starting material. Upon completion, reaction mixtures were concentrated in vacuo, then treated with CH₂Cl₂ and concentrated twice to remove residual TFA. The crude solids were triturated with Et₂O to afford the compounds as TFA salts.

### Experimental Procedures and Tabulated Characterization Data

### (Dipeptide) methyl N^{α}-((benzyloxy)carbonyl)-NE-(tert-butoxycarbonyl)-D-lysyl-L-valinate (1)

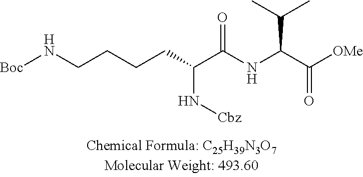 [SMILES: COC(=O)[C@@H](NC(=O)[C@@H](CCCCNC(=O)OC(C)(C)C)NC(=O)OCc1ccccc1)C(C)C]

Dipeptide 1 was prepared using the general procedure for peptide coupling with Cbz-D-Lys(Boc)-OH (3.75 g, 9.0 mmol, 1.0 eq), CH₂Cl₂ (65 mL, 0.1 M), HATU (4.13 g, 10.9 mmol, 1.1 eq), DIEA (5.15 mL, 29.6 mmol, 3.0 eq) and H-L-Val-OMe-HCl (1.65 g, 9.9 mmol, 1.0 eq) and a total reaction time of 13.5 hours. The crude product was recrystallized from hot EtOAc, isolated by filtration, and washed with hexanes to afford the pure compound 1 as a white solid (3.74 g, 66% yield). ¹H NMR (400 MHz, CDCl₃) δ 7.28-7.15 (m, 5H), 6.57 (d, J=8.6 Hz, 1H), 5.50-5.46 (m, 1H), 5.02 (dd, J=22.9, 12.3 Hz, 2H), 4.53 (s, 1H), 4.42 (dd, J=8.8, 4.9 Hz, 1H), 4.15-4.09 (m, 1H), 3.62 (s, 3H), 3.05-2.95 (m, 2H), 2.12-1.99 (m, 1H), 1.85-1.72 (m, 1H), 1.65-1.51 (m, 1H), 1.45-1.24 (m, 13H), 0.80 (dd, J=20.5, 6.8 Hz, 6H); ¹³C NMR (126 MHz, CDCl₃) δ 172.29, 171.83, 156.44, 156.33, 136.35, 128.63, 128.30, 128.24, 79.31, 67.19, 57.20, 55.10, 52.27, 39.86, 32.16, 31.26, 29.78, 28.52, 22.53, 19.08, 17.83; IR (solid) νₘₐₓ 3334, 3315, 2958, 2937, 2856, 1734, 1684, 1651, 1531, 1460, 1390, 1365, 1321, 1267, 1246, 1172, 1059, 1018, 852, 779 cm⁻¹; HRMS (ESMS) calculated for C₂₅H₃₉N₃O₇⁺ [M+H]⁺, 494.2861, found: 494.2856; [α]_{D}_{²⁰}=+38 (c=0.9, CHCl₃).

### (Tripeptide) methyl N²-((benzyloxy)carbonyl)-N⁶-((tert-butoxycarbonyl)-L-phenylalanyl)-D-lysyl-L-valinate (2)

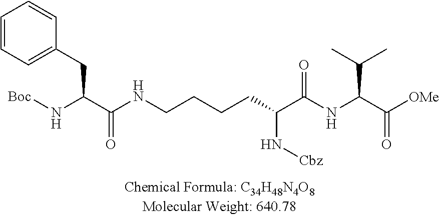 [SMILES: COC(=O)[C@@H](NC(=O)[C@@H](CCCCNC(=O)[C@H](Cc1ccccc1)NC(=O)OC(C)(C)C)NC(=O)OCc1ccccc1)C(C)C]

Dipeptide 1 was subjected to Boc deprotection using the general procedure and Cbz-D-Lys(Boc)-Val-OMe (6.05 g, 12.3 mmol, 1.0 eq), CH₂Cl₂ (82 mL, 0.15 M) and TFA (22 mL) and a total reaction time of 1 h. The salt Cbz-D-Lys-Val-OMe-TFA was used without further purification. Tripeptide 2 was prepared using Boc-Phe-OH (3.43 g, 12.9 mmol, 1.0 eq), CH₂Cl₂ (86 mL, 0.15 M) HATU (5.41 g, 14.2 mmol, 1.1 eq), DIEA (6.8 mL, 38.8 mmol, 3.0 eq), and Cbz-D-Lys-Val-OMe-TFA salt (6.56 g, 12.9 mmol, 1.0 eq) with a total reaction time of 17 h. The crude product was recrystallized from hot EtOAc, isolated by filtration, and washed with hexanes to afford the pure compound 2 as a white solid (5.59 g, 67% yield—collected from three crops). ¹H NMR (400 MHz, CDCl₃) δ 7.40-7.26 (m, 7H), 7.27-7.15 (m, 3H), 6.60 (d, J=7.3 Hz, 1H), 5.87 (t, 1H), 5.49 (s, 1H), 5.13 (m, 3H), 4.50 (dd, J=8.8, 4.9 Hz, 1H), 4.30-4.23 (m, 1H), 4.17-4.11 (m, 1H), 3.72 (s, 3H), 3.19-3.13 (m, 2H), 3.09-2.95 (m, 2H), 2.19-2.13 (m, 1H), 1.89-1.77 (m, 1H), 1.62 (m, 1H), 1.40 (s, 11H), 1.33-1.21 (m, 2H), 0.89 (dd, J=21.8, 6.7 Hz, 6H). ¹³C NMR (126 MHz, CDCl₃) δ 172.38, 171.99, 171.61, 156.50, 155.62, 137.08, 136.33, 129.42, 128.68, 128.64, 128.29, 128.23, 126.94, 80.16, 67.19, 57.28, 56.11, 55.04, 52.27, 38.86, 38.82, 31.93, 31.20, 28.94, 28.38, 22.58, 19.10, 17.88. IR (solid) νₘₐₓ 3319, 2970, 2941, 1736, 1689, 1651, 1529, 1439, 1390, 1369, 1273, 1248, 1171, 1047, 1024, 754, 696 cm⁻¹; HRMS (ESMS) calculated for C₃₄H₄₈N₄O₈⁺ [M+H]⁺, 641.3545, found: 641.3543; [α]_{D}_{²⁰}=+12 (c=0.9, CHCl₃).

### (Tetrapeptide) methyl N²-((benzyloxy)carbonyl)-N⁶—N-(tert-butoxycarbonyl)-N-methyl-L-alanyl-L-phenylalanyl-D-lysyl-L-valinate (3)

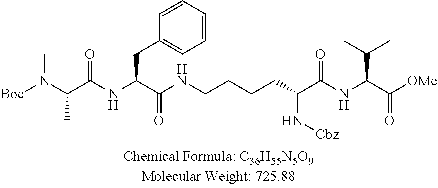 [SMILES: COC(=O)[C@@H](NC(=O)[C@@H](CCCCNC(=O)[C@H](Cc1ccccc1)NC(=O)[C@H](C)N(C)C(=O)OC(C)(C)C)NC(=O)OCc1ccccc1)C(C)C]

Tripeptide 2 was subjected to Boc deprotection using the general procedure, and Cbz-D-Lys(Boc-Phe)-Val-OMe (2.50 g, 3.9 mmol, 1.0 eq), CH₂Cl₂ (26 mL, 0.15 M), TFA (7 mL) and a total reaction time of 1.5 h. The salt Cbz-D-Lys(H-Phe)-Val-OMe-TFA was used without further purification. Tetrapeptide 3 was prepared using Boc-MeAla-OH (0.79 g, 3.9 mmol, 1.0 eq), CH₂Cl₂ (26 mL, 0.15 M), HATU (1.63 g, 4.3 mmol, 1.1 eq), DIEA (2.0 mL, 11.7 mmol, 3.0 eq), and the salt Cbz-D-Lys(Boc-Phe)-Val-OMe-TFA (2.56 g, 3.9 mmol, 1.0 eq) and a total reaction time of 18 h. The crude was purified by flash column chromatography (75% EtOAc/Hx) to afford 3 as a white foam (2.33 g, 82% yield). ¹H NMR (500 MHz, CDCl₃) δ 7.38-7.27 (m, 6H), 7.25-7.21 (m, 2H), 7.16 (d, J=7.3 Hz, 2H), 6.69 (d, J=8.7 Hz, 1H), 6.43 (s, 1H), 5.63 (s, 1H), 5.12 (dd, J=24.9, 12.1 Hz, 2H), 4.63-4.46 (m, 3H), 4.23-4.09 (m, 1H), 3.72 (s, 3H), 3.26-3.11 (m, 2H), 3.06 (m, 2H), 2.55 (s, 3H), 2.21-2.10 (m, 1H), 1.89-1.78 (m, 1H), 1.70-1.58 (m, 1H), 1.51-1.38 (m, 12H), 1.35-1.27 (m, 2H), 1.25 (d, J=6.9 Hz, 3H), 0.92 (d, J=5.1 Hz, 3H), 0.86 (d, J=6.4 Hz, 3H). IR (solid) νₘₐₓ 3294, 2960, 2924, 2860, 1732, 1649, 1533, 1454, 1437, 1390, 1367, 1315, 1248, 1147, 1028, 987, 912 cm⁻¹; HRMS (ESMS) calculated for C₃₈H₅₅N₅O₉⁺ [M+H]⁺, 726.4037, found: 726.4060; [α]_{D}_{²⁰}=−19 (c 0.77, MeOH).

### (Boc-Hty(Oallyl)-OH (S)-4-(4-(allyloxy)phenyl)-2-((tert-butoxycarbonyl)amino) butanoic acid (4)

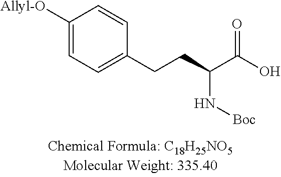

Boc-HTyr-OH (1.50 g, 5.1 mmol, 1.0 eq) was treated with K₂CO₃ (3.51 g, 25.4 mmol, 5.0 eq) and 3-Bromopropene (1.33 mL, 15.2 mmol, 3.0 eq) in dry DMF (33 mL, 0.15 M) with a total reaction time of 38 h. The solution was quenched with water and extracted with EtOAc. The organics were washed with water, dried over Na₂SO₄, filtered, and concentrated in vacuo. The crude was purified by flash column chromatography (10-25% EtOAc/Hx) to afford a yellow oil (1.46 g, 77% yield). Bis-allyl Homotyrosine (Bis Allyl Hty) (1.44 g, 3.84 mmol, 1.0 eq) was treated with LiOH (0.17 g, 6.91 mmol, 1.8 eq) in THF/Water (5:1, 25 mL, 0.15 M) with a total reaction time of 2.5 h. The mixture was concentrated, diluted with pH 3 water acidified with citric acid monohydrate, and extracted with EtOAc. The organics were dried over Na₂SO₄, filtered, and concentrated in vacuo to afford 4 as a yellow oil (1.29 g, 100% yield). ¹H NMR (500 MHz, MeOD) δ 7.10 (d, J=8.6 Hz, 2H), 6.84 (d, J=8.4 Hz, 2H), 6.04 (ddt, J=17.3, 10.5, 5.2 Hz, 1H), 5.38 (dq, J=17.3, 1.7 Hz, 1H), 5.23 (dq, J=10.6, 1.6 Hz, 1H), 4.50 (dt, J=5.1, 1.7 Hz, 2H), 4.05 (dd, J=9.4, 4.6 Hz, 1H), 2.63 (m, 2H), 2.11-2.00 (m, 1H), 1.94-1.83 (m, 1H), 1.46 (m, 9H). ¹³C NMR (126 MHz, MeOD) δ 176.27, 158.48, 158.15, 135.11, 134.54, 130.44, 117.29, 115.79, 80.47, 69.81, 54.32, 34.87, 32.18, 28.75. HRMS (ESMS) calculated for C₁₈H₂₅NO₅⁻ [M−H]⁻, 334.1660, found: 334.1613; [α]_{D}_{²⁰}=+38 (c=1.1, CHCl₃).

### (Pentapeptide) methyl N⁶—N—((S)-4-(4-(allyloxy)phenyl)-2-((tert-butoxycarbonyl)amino)butanoyl)-N-methyl-L-alanyl-L-phenylalanyl-N²-((benzyloxy)carbonyl)-D-lysyl-L-valinate (5)

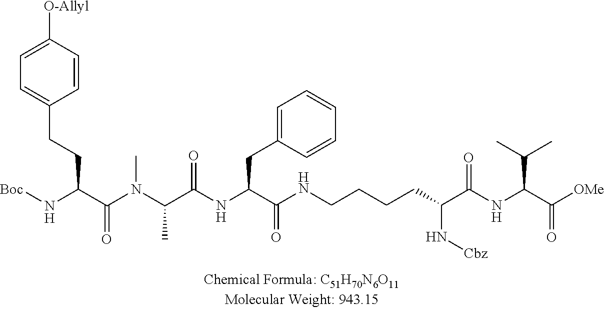

Tetrapeptide 3 was subjected to Boc deprotection using the general procedure and Cbz-D-Lys(Boc-MeAla-Phe)-Val-OMe (30.0 mg, 0.42 mmol, 1.0 eq), CH₂Cl₂ (4 mL, 0.1 M) and TFA (1 mL) and a total reaction time of 1.5 h. The salt Cbz-D-Lys(H-MeAla-Phe)-Val-OMe-TFA was used without further purification. Pentapeptide 5 was prepared using Boc-HTyr(O-Allyl)-OH (1.08 g, 3.2 mmol, 1.0 eq), CH₂Cl₂ (21 mL, 0.15 M), HATU (1.34 g, 3.5 mmol, 1.1 eq), DIEA (1.7 mL, 9.6 mmol, 3.0 eq), Cbz-D-Lys(H-MeAla-Phe)-Val-OMe-TFA salt (2.37 g, 3.2 mmol, 1.0 eq) and a total reaction time of 13.5 h. The crude was purified by flash column chromatography (80% EtOAc/Hx) to afford 5 as a white foam (2.69 g, 89% yield). The compound exists as a mixture of rotamers. Diagnostic data is as follows; ¹H NMR (500 MHz, MeOD) δ 7.38-7.23 (m, 6H), 7.23-7.07 (m, 6H), 6.89-6.81 (m, 2H), 6.03 (ddq, J=17.3, 10.0, 5.0 Hz, 1H), 5.37 (ddq, J=17.3, 3.5, 1.8 Hz, 1H), 5.22 (dq, J=10.6, 1.6 Hz, 1H), 3.69 (s, 3H), 0.90 (dd, J=9.1, 6.7 Hz, 6H); ¹³C NMR (126 MHz, MeOD) δ 175.17, 173.64, 158.61, 135.07, 129.49, 67.74, 28.99, 19.53; IR (solid) νₘₐₓ 3300, 2966, 2931, 2866, 1705, 1651, 1525, 1510, 1454, 1439, 1408, 1390, 1365, 1240, 1167, 1113, 1045, 1024, 997, 924, 845, 741 cm⁻¹; HRMS (ESMS) calculated for C₅₁H₇₀N₆O₁₁⁺ [M+H]⁺, 943.5175, found: 943.5157; [α]_{D}_{²⁰}=+27 (c=0.8, CHCl₃).

### (Pentacycle Protected) benzyl ((3S,6S,9S,12S,15R)-9-(4-(allyloxy)phenethyl)-3-benzyl-12-isopropyl-6,7-dimethyl-2,5,8,11,14-pentaoxo-1,4,7,10,13-pentaazacyclononadecan-15-yl)carbamate (6)

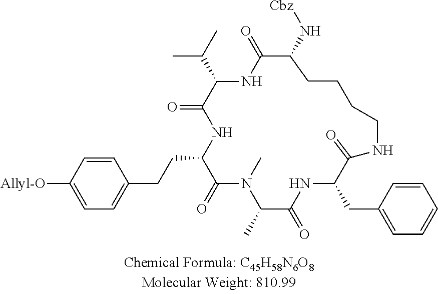

Pentapeptide 5 (1.78 g, 1.88 mmol, 1.0 eq) was treated with LiOH (5.09 mmol, 2.7 eq) in THF/Water (5:1, 19 mL, 0.1 M) with a total reaction time of 4 hours. The reaction was concentrated, diluted with pH 3 water (acidified with citric acid monohydrate), and extracted with EtOAc (3×50 mL). The organic layer was dried over anhydrous Na₂SO₄, filtered, and concentrated in vacuo to afford (5.1?) as a white solid/foam (1.71 g, 98% yield). (5.1?) was subjected to Boc deprotection using the general procedure and Cbz-D-Lys(Boc-HTyr(O-allyl)-MeAla-Phe)-Val-OH (1.70 g, 1.83 mmol, 1.0 eq), CH₂Cl₂ (18 mL, 0.1 M), and TFA (3.5 mL), and a total reaction time of 2 h. The salt Cbz-D-Lys(H-HTyr(O-Allyl)-MeAla-Phe)-Val-OMe-TFA was used without further purification. The protected pentacycle 6 was prepared by dissolving the salt Cbz-D-Lys(H-HTyr(O-Allyl)-MeAla-Phe)-Val-OH-TFA (0.99 g, 1.0 mmol, 1.0 eq) in CH₂Cl₂ (100 mL) and adding dropwise by an addition funnel into a solution of HATU (1.19 g, 3.1 mmol, 3.0 eq), DIEA (0.73 mL, 4.2 mmol, 4.0 eq) and CH₂Cl₂ (1046 mL, 0.001 M—with respect to the peptide). The reaction was worked up after 46 h. The crude was purified by flash column chromatography (75-100% EtOAc/Hx) to afford 6 as a white foam (0.60 g, 70% yield). ¹H NMR (500 MHz, MeOD) δ 7.39-7.26 (m, 5H), 7.23 (dd, J=8.0, 6.7 Hz, 2H), 7.20-7.13 (m, 3H), 7.11 (d, J=7.2 Hz, 2H), 6.89-6.82 (m, 2H), 6.03 (ddt, J=17.3, 10.4, 5.1 Hz, 1H), 5.36 (dq, J=17.2, 1.7 Hz, 1H), 5.22 (dq, J=10.5, 1.5 Hz, 1H), 5.17-5.02 (m, 2H), 4.77-4.69 (m, 2H), 4.60 (dd, J=12.6, 3.4 Hz, 1H), 4.51 (dt, J=5.2, 1.6 Hz, 2H), 4.10 (dd, J=7.1, 4.6 Hz, 1H), 3.91 (d, J=9.3 Hz, 1H), 3.68 (dt, J=13.0, 6.1 Hz, 1H), 3.38 (dd, J=14.0, 3.5 Hz, 1H), 2.92 (dt, J=13.6, 4.9 Hz, 1H), 2.87-2.74 (m, 2H), 2.65-2.55 (m, 1H), 2.12-2.04 (m, 2H), 1.86 (s, 5H), 1.79-1.69 (m, 1H), 1.62-1.52 (m, 2H), 1.49-1.36 (m, 2H), 1.30 (m, 3H), 1.15 (t, J=6.5 Hz, 6H), 0.98 (d, J=6.5 Hz, 3H). IR (solid) νₘₐₓ 3286, 2962, 2933, 2870, 1724, 1645, 1510, 1454, 1439, 1390, 1367, 1317, 1238, 1153, 1080, 1026 cm⁻¹; HRMS (ESMS) calculated for C₄₅H₅₈N₆O₈⁺ [M+H]⁺ 811.4389, found: 811.4358; [α]_{D}_{²⁰}=−67 (c=0.68, MeOH).

### Pentacycle Deprotected) (3S,6S,9S,12S,15R)-15-amino-3-benzyl-9-(4-hydroxyphenethyl)-12-isopropyl-6,7-dimethyl-1,4,7,10,13-pentaazacyclononadecane-2,5,8,11,14-pentaone (7)

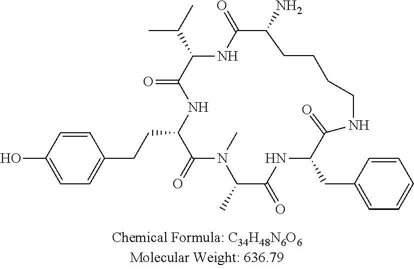 [SMILES: CC(C)[C@@H]1NC(=O)[C@H](N)CCCCNC(=O)[C@H](Cc2ccccc2)NC(=O)[C@H](C)N(C)C(=O)[C@H](CCc2ccc(O)cc2)NC1=O]

The pentacycle salt 6.1 was prepared using protected pentacycle (208 mg, 0.257 mmol, 1.0 eq), 33 wt % HBr in AcOH (1.5 mL). After 2 h, the black solution was added dropwise to a stirring flask of Et₂O (20 mL), filtered through a 2 mL fine-fritted filter, and washed with Et₂O (5 mL×5) to afford the intermediate 6.1 as a light gray powdered solid which was used without further purification. The pentacycle salt 6.1 was treated with 10% NH₄OH (2.5 mL) and MeOH (10 mL), stirred for 2 h, and concentrated in vacuo. The crude was purified by flash column chromatography (1-10% MeOH/DCM with 1% NH₄OH) to afford 7 as a white solid (110 mg, 67% yield over two steps). ¹H NMR (500 MHz, MeOD) δ 8.83 (d, J=8.7 Hz, 1H), 7.23 (dd, J=8.1, 6.7 Hz, 2H), 7.19-7.10 (m, 3H), 7.07 (d, J=8.5 Hz, 2H), 6.71 (d, J=8.5 Hz, 2H), 4.76-4.66 (m, 2H), 4.63-4.57 (m, 1H), 4.02 (d, J=8.7 Hz, 1H), 3.69 (ddd, J=13.1, 7.7, 5.1 Hz, 1H), 3.52 (dd, J=6.7, 3.8 Hz, 1H), 3.39 (dd, J=14.0, 3.5 Hz, 1H), 2.93 (dt, J=13.6, 5.2 Hz, 1H), 2.84 (dd, J=14.0, 12.7 Hz, 1H), 2.75 (ddd, J=13.7, 9.3, 4.4 Hz, 1H), 2.57 (dt, J=13.7, 8.5 Hz, 1H), 2.22-2.12 (m, 1H), 2.07 (dtd, J=13.6, 9.1, 4.4 Hz, 1H), 1.88 (s, 3H), 1.83 (ddt, J=17.1, 13.5, 7.2 Hz, 2H), 1.68 (ddt, J=13.6, 9.0, 6.8 Hz, 1H), 1.56 (ddq, J=12.1, 8.2, 4.7 Hz, 2H), 1.41-1.26 (m, 2H), 1.18 (d, J=6.8 Hz, 3H), 1.14 (t, J=6.7 Hz, 6H); ¹³C NMR (126 MHz, MeOD) δ 178.28, 174.71, 174.10, 173.87, 172.24, 157.00, 139.11, 132.78, 130.53, 130.14, 129.74, 127.65, 116.33, 60.47, 57.06, 56.80, 56.44, 50.23, 40.08, 38.85, 35.25, 34.64, 32.10, 31.75, 29.54, 28.31, 21.39, 19.97, 19.86, 14.29; [α]_{D}_{²⁰}=−78 (c=0.53, MeOH).

### Anabaenopeptin A (8)

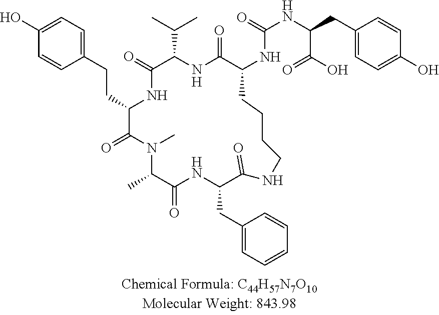 [SMILES: CC(C)[C@@H]1NC(=O)[C@H](NC(=O)N[C@@H](Cc2ccc(O)cc2)C(=O)O)CCCCNC(=O)[C@H](Cc2ccccc2)NC(=O)[C@H](C)N(C)C(=O)[C@H](CCc2ccc(O)cc2)NC1=O]

H-Tyr(O-tBu)-OtBu-HCl salt (200 mg, 606 μmol, 1.0 eq), triethylamine (170 μL, 1.2 mmol, 2.0 eq), and CH₂Cl₂ (4 mL, 0.15 M) were stirred, then 4-nitrophenyl chloroformate (134 mg, 667 μmol, 1.1 eq) was added and the reaction was stirred for 15 h. The reaction was concentrated, brought up in EtOAc, and washed with NaHCO₃ (50 mL×3), pH 3 water (acidified with citric acid monohydrate) (50 mL), and brine (50 mL). The organic layer was dried over Na₂SO₄, filtered, and concentrated in vacuo to afford (7.1) as a light yellow/colorless oil which was used without further purification.

(7.1) (18.0 mg, 39.3 μmol, 1.0 eq) and (7) (25.0 mg, 39.3 μmol, 1.0 eq) were dissolved in anhydrous DMF (440 μL, 0.1 M), and DIEA (13 μL, 78.5 μmol, 2.0 eq) was added dropwise. The solution was stirred for 16 hours at room temperature. The reaction was diluted with EtOAc, washed with saturated NaHCO₃, NH₄Cl, and brine. The organic layer was dried over Na₂SO₄, filtered, and concentrated in vacuo. The crude was loaded onto a prep plate with DCM/MeOH and developed in a chamber with 4% MeOH/EtOAc. The band of silica was isolated, extracted with 10% MeOH/DCM, and the compound was isolated by filtration through a course fritted filter to afford (7.2) as a white solid (24.1 mg 64% yield). (7.2) (24.1 mg, 3 μmol, 1.0 eq) was subjected to t-butyl deprotection by treating with TFA/DCM (1:1, 2.5 ml) and a total reaction time of 18 h. The reaction was concentrated in vacuo, to afford (8) as a white solid (14.4 mg, 68% yield). ¹H NMR (600 MHz, DMSO) δ 9.18 (d, J=13.7 Hz, 2H), 8.91 (d, J=4.7 Hz, 1H), 8.65 (d, J=8.9 Hz, 1H), 7.18 (t, J=7.4 Hz, 2H), 7.13 (dd, J=8.8, 6.1 Hz, 2H), 7.02-6.90 (m, 5H), 6.65 (t, J=8.8 Hz, 5H), 6.50 (d, J=7.2 Hz, 1H), 6.18 (d, J=8.3 Hz, 1H), 4.77 (d, J=7.1 Hz, 1H), 4.74-4.68 (m, 1H), 4.41-4.33 (m, 1H), 4.24 (d, J=8.4 Hz, 1H), 3.90 (p, J=7.1 Hz, 2H), 3.29 (s, 18H), 2.86 (dd, J=13.8, 5.2 Hz, 1H), 2.82-2.71 (m, 3H), 2.49-2.38 (m, 1H), 1.93 (dp, J=13.7, 6.7 Hz, 1H), 1.76 (s, 3H), 1.66-1.53 (m, 2H), 1.42 (d, J=18.2 Hz, 2H), 1.12 (d, J=8.2 Hz, OH), 1.04 (dd, J=17.8, 6.6 Hz, 7H), 0.92 (d, J=6.5 Hz, 3H). ¹³C NMR ¹³C NMR (151 MHz, DMSO) δ 173.79, 172.58, 172.17, 170.91, 170.78, 169.81, 157.01, 155.89, 155.52, 138.24, 130.99, 130.12, 129.02, 128.83, 128.27, 126.07, 115.11, 114.93, 69.76, 58.10, 54.88, 54.62, 54.24, 54.15, 48.67, 38.27, 37.49, 36.75, 33.22, 31.75, 30.48, 30.00, 28.08, 26.98, 20.28, 19.23, 18.89, 13.80. OR [α]_{D}²⁰=−61 (c=0.7, CHCl₃). Purity Analysis. The crude was subjected to LCMS analysis to measure purity, with measurements taken at 254 nm and 220 nm. The UV spectrum of the crude revealed three absorbance peaks, with the highest peak observed at 277 nm. The LC chromatogram and mass spectrum indicated the presence of one impurity eluting prior to the product, constituting 3%, while the desired product comprised the major component at 97%.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A compound having formula 1: 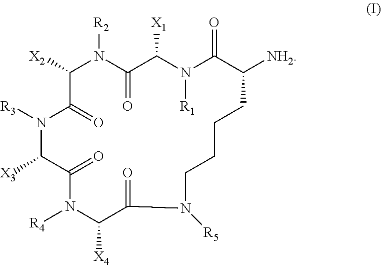
or a salt thereof and/or stereoisomers thereof and/or isotopic variations thereof,
wherein:
X₁, X₂, X₃, and X₄ are alkyl, aryl, hydroxyl, alkyl phenyl, alkyl phenol, and benzyl groups; and
R₁, R₂, R₃, R₄, and R₅ are H or C₁₋₁₀ alkyl, wherein at least one of X₁, X₂, X₃, and X₄ is alkyl phenol, with the proviso that at least one of X₁, X₂, X₃, and X₄ are alkyl phenol.

2. The compound of claim 1, wherein at least two of X₁, X₂, X₃, and X₄ are alkyl phenol.

3. The compound of claim 1, wherein the alkyl phenol is

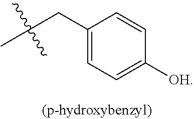 [SMILES: *Cc1ccc(O)cc1]

4. The compound of claim 1, wherein X₁ is isopropyl.

5. The compound of claim 1, wherein X₂ and/or X₃ is p-hydroxybenzyl.

6. The compound of claim 1, wherein X₃ is methyl.

7. The compound of claim 1, wherein X₄ is benzyl.

8. The compound of claim 1, wherein X₁ is isopropyl, X₂ is p-hydroxybenzyl, X₃ is methyl, and X₄ is benzyl.

9. The compound of claim 1, wherein 1 to 3 of R₁, R₂, R₃, R₄, and R₅ are H, methyl, ethyl, propyl, isopropyl, butyl, isobutyl, sec-butyl, tert-butyl, pentyl, isopentyl, neopentyl, hexyl, or isohexyl.

10. The compound of claim 1, wherein at least one of X₁, X₂, X₃, and X₄ are not one of hydroxymethyl, hydroxyethyl, thiol, thioether, p-hydroxybenzyl, indolylmethyl, carboxymethyl, carboxyethyl, carboxamide, carboxyethylamide, butylammonium, guanidinium, imidazolylmethyl, or pyrrolidine.

11. The compound of claim 1 having formula II: 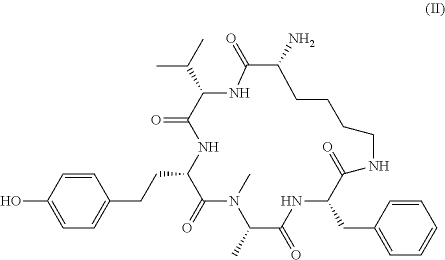 [SMILES: CC(C)[C@@H]1NC(=O)[C@H](N)CCCCNC(=O)[C@H](Cc2ccccc2)NC(=O)[C@H](C)N(C)C(=O)[C@H](CCc2ccc(O)cc2)NC1=O]
or a salt thereof and/or stereoisomers thereof and/or isotopic variations thereof.

12. The compound of claim 1, wherein the isotopic variations include
hydrogen isotopes selected from the group consisting of protium (¹H), deuterium (²H), tritium (³H), and combinations thereof; and/or
carbon isotopes selected from the group consisting of carbon-12 (¹²C), carbon-13 (₁₃C), carbon-14 (¹⁴C), and combinations thereof; and/or
oxygen isotopes selected from the group consisting of oxygen-16 (¹⁶O), oxygen-17 (¹⁷O), oxygen-18 (¹⁸O), and combinations thereof; and/or
nitrogen isotopes selected from the group consisting of nitrogen-14 (¹⁴N), nitrogen-15 (¹⁵N), and combinations thereof.

13. A method for making the compound of claim 1 comprising preparing a pentapeptide that includes residues of amino acids having the following formula: 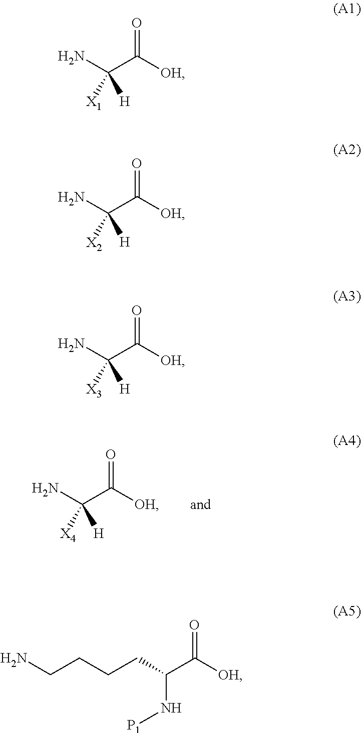
with the proviso that at least one of X₁, X₂, X₃, and X₄ are alkyl phenol; and
cyclizing the pentapeptide to form the compound having formula (I).

14. The method of claim 13, wherein the pentapeptide is selected form the group consisting of H₂N-A1-A2-A3-A4-A5-CO₂H; H₂N-A1-A2-A3-A5-A4-CO₂H; H₂N-A1-A2-A4-A3-A5-CO₂H; NH₂-A1-A2-A4-A5-A3-CO₂H; A1-A2-A5-A3-A4-CO₂H; NH₂-A1-A2-A5-A4-A3-CO₂H; NH₂-A1-A3-A2-A4-A5-CO₂H; NH₂-A1-A3-A2-A5-A4-CO₂H; NH₂-A1-A3-A4-A2-A5-CO₂H; NH₂-A1-A3-A4-A5-A2-CO₂H; NH₂-A1-A3-A5-A2-A4-CO₂H; NH₂-A1-A3-A5-A4-A2-CO₂H; NH₂-A1-A4-A2-A3-A5-CO₂H; NH₂-A1-A4-A2-A5-A3-CO₂H; NH₂-A1-A4-A3-A2-A5-CO₂H; NH₂-A1-A4-A3-A5-A2-CO₂H; NH₂-A1-A4-A5-A2-A3-CO₂H; NH₂-A1-A4-A5-A3-A2-CO₂H; NH₂-A1-A5-A2-A3-A4-CO₂H; NH₂-A1-A5-A2-A4-A3-CO₂H; NH₂-A1-A5-A3-A2-A4-CO₂H; NH₂-A1-A5-A3-A4-A2-CO₂H; NH₂-A1-A5-A4-A2-A3-CO₂H; NH₂-A1-A5-A4-A3-A2-CO₂H; NH₂-A2-A1-A3-A4-A5-CO₂H; NH₂-A2-A1-A3-A5-A4-CO₂H; NH₂-A2-A1-A4-A3-A5-CO₂H; NH₂-A2-A1-A4-A5-A3-CO₂H; NH₂-A2-A1-A5-A3-A4-CO₂H; NH₂-A2-A1-A5-A4-A3-CO₂H; NH₂-A2-A3-A1-A4-A5-CO₂H; NH₂-A2-A3-A1-A5-A4-CO₂H; NH₂-A2-A3-A4-A1-A5-CO₂H; NH₂-A2-A3-A4-A5-A1-CO₂H; NH₂-A2-A3-A5-A1-A4-CO₂H; NH₂-A2-A3-A5-A4-A1-CO₂H; NH₂-A2-A4-A1-A3-A5-CO₂H; NH₂-A2-A4-A1-A5-A3-CO₂H; NH₂-A2-A4-A3-A1-A5-CO₂H; NH₂-A2-A4-A3-A5-A1-CO₂H; NH₂-A2-A4-A5-A1-A3-CO₂H; NH₂-A2-A4-A5-A3-A1-CO₂H; NH₂-A2-A5-A1-A3-A4-CO₂H; NH₂-A2-A5-A1-A4-A3-CO₂H; NH₂-A2-A5-A3-A1-A4-CO₂H; NH₂-A2-A5-A3-A4-A1-CO₂H; NH₂-A2-A5-A4-A1-A3-CO₂H; NH₂-A2-A5-A4-A3-A1-CO₂H; NH₂-A3-A1-A2-A4-A5-CO₂H; NH₂-A3-A1-A2-A5-A4-CO₂H; NH₂-A3-A1-A4-A2-A5-CO₂H; NH₂-A3-A1-A4-A5-A2-CO₂H; NH₂-A3-A1-A5-A2-A4-CO₂H; NH₂-A3-A1-A5-A4-A2-CO₂H; NH₂-A3-A2-A1-A4-A5-CO₂H; NH₂-A3-A2-A1-A5-A4-CO₂H; NH₂-A3-A2-A4-A1-A5-CO₂H; NH₂-A3-A2-A4-A5-A1-CO₂H; NH₂-A3-A2-A5-A1-A4-CO₂H; NH₂-A3-A2-A5-A4-A1-CO₂H; NH₂-A3-A4-A1-A2-A5-CO₂H; NH₂-A3-A4-A1-A5-A2-CO₂H; NH₂-A3-A4-A2-A1-A5-CO₂H; NH₂-A3-A4-A2-A5-A1-CO₂H; NH₂-A3-A4-A5-A1-A2-CO₂H; NH₂-A3-A4-A5-A2-A1-CO₂H; NH₂-A3-A5-A1-A2-A4-CO₂H; NH₂-A3-A5-A1-A4-A2-CO₂H; NH₂-A3-A5-A2-A1-A4-CO₂H; NH₂-A3-A5-A2-A4-A1-CO₂H; NH₂-A3-A5-A4-A1-A2-CO₂H; NH₂-A3-A5-A4-A2-A1-CO₂H; NH₂-A4-A1-A2-A3-A5-CO₂H; NH₂-A4-A1-A2-A5-A3-CO₂H; NH₂-A4-A1-A3-A2-A5-CO₂H; NH₂-A4-A1-A3-A5-A2-CO₂H; NH₂-A4-A1-A5-A2-A3-CO₂H; NH₂-A4-A1-A5-A3-A2-CO₂H; NH₂-A4-A2-A1-A3-A5-CO₂H; NH₂-A4-A2-A1-A5-A3-CO₂H; NH₂-A4-A2-A3-A1-A5-CO₂H; NH₂-A4-A2-A3-A5-A1-CO₂H; NH₂-A4-A2-A5-A1-A3-CO₂H; NH₂-A4-A2-A5-A3-A1-CO₂H; NH₂-A4-A3-A1-A2-A5-CO₂H; NH₂-A4-A3-A1-A5-A2-CO₂H; NH₂-A4-A3-A2-A1-A5-CO₂H; NH₂-A4-A3-A2-A5-A1-CO₂H; NH₂-A4-A3-A5-A1-A2-CO₂H; NH₂-A4-A3-A5-A2-A1-CO₂H; NH₂-A4-A5-A1-A2-A3-CO₂H; NH₂-A4-A5-A1-A3-A2-CO₂H; NH₂-A4-A5-A2-A1-A3-CO₂H; NH₂-A4-A5-A2-A3-A1-CO₂H; NH₂-A4-A5-A3-A1-A2-CO₂H; NH₂-A4-A5-A3-A2-A1-CO₂H; NH₂-A5-A1-A2-A3-A4-CO₂H; NH₂-A5-A1-A2-A4-A3-CO₂H; NH₂-A5-A1-A3-A2-A4-CO₂H; NH₂-A5-A1-A3-A4-A2-CO₂H; NH₂-A5-A1-A4-A2-A3-CO₂H; NH₂-A5-A1-A4-A3-A2-CO₂H; NH₂-A5-A2-A1-A3-A4-CO₂H; NH₂-A5-A2-A1-A4-A3-CO₂H; NH₂-A5-A2-A3-A1-A4-CO₂H; NH₂-A5-A2-A3-A4-A1-CO₂H; NH₂-A5-A2-A4-A1-A3-CO₂H; NH₂-A5-A2-A4-A3-A1-CO₂H; NH₂-A5-A3-A1-A2-A4-CO₂H; NH₂-A5-A3-A1-A4-A2-CO₂H; NH₂-A5-A3-A2-A1-A4-CO₂H; NH₂-A5-A3-A2-A4-A1-CO₂H; NH₂-A5-A3-A4-A1-A2-CO₂H; NH₂-A5-A3-A4-A2-A1-CO₂H; NH₂-A5-A4-A1-A2-A3-CO₂H; NH₂-A5-A4-A1-A3-A2-CO₂H; NH₂-A5-A4-A2-A1-A3-CO₂H; NH₂-A5-A4-A2-A3-A1-CO₂H; NH₂-A5-A4-A3-A1-A2-CO₂H; and NH₂-5-A4-A3-A2-A1 where “-” between any 2 of A1, A2, A3, A4, and A5-represents a peptide bond.

15. A method for making the compound of claim 1 comprising:
a) providing a first protected amino acid having formula P1 or a salt thereof and/or stereoisomers thereof and/or isotopic variations thereof: 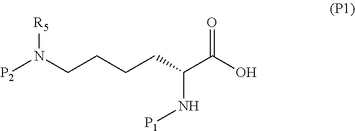
wherein P₁ is a first protecting group and P₂ is a second protecting group that can be selectively removed under different conditions;
b) attaching a second protected amino acid to the C-terminus side of the first protected amino acid having formula P1 and then removing protecting group P₂ to form a peptide having formula P2 or a salt thereof and/or stereoisomers thereof and/or isotopic variations thereof with an exposed amino group: 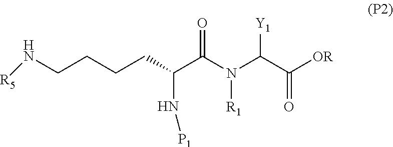
wherein Y₁ is X₁ or X₁ with a removable protecting group and OR is a C₁₋₆ alkoxyl protecting group;
c) attaching a third protected amino acid to the exposed amino group to form a peptide having formula P3 or a salt thereof and/or stereoisomers thereof and/or isotopic variations thereof: 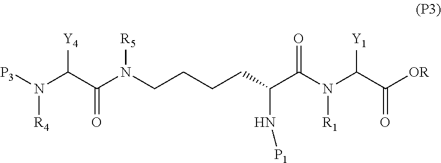
wherein Y₄ is X₄ or a X₄ with a removable protecting group and P₃ is a third protecting group;
d) removing P₄ and attaching a fourth protected amino acid to the N-terminus side of the peptide having formula P3 to form a peptide having formula P4 or a salt thereof and/or stereoisomers thereof and/or isotopic variations thereof: 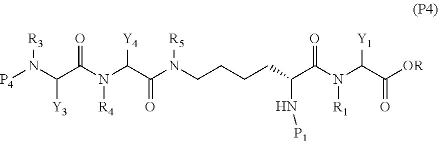
wherein Y₃ is X₃ or a X₃ with a removable protecting group and P₄ is a fourth protecting group;
e) removing P₄ and attaching fifth protected amino acid to the N-terminus side of the peptide having formula 34 to form a peptide having formula 5 a salt thereof and/or stereoisomers thereof and/or isotopic variations thereof: 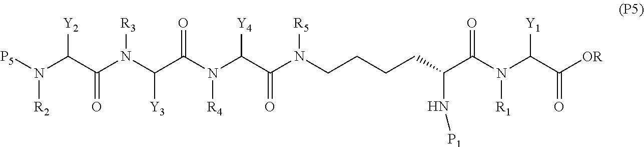
wherein Y₂ is X₂ or a X₂ with a removable protecting group and P₅ is a fifth protecting group;
f) removing OR and P₅ to form pentapeptide P6 or a salt thereof and/or stereoisomers thereof and/or isotopic variations thereof: 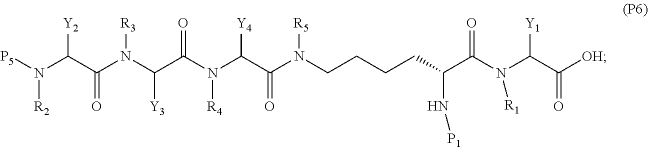
and
g) cyclizing the peptide having formula P5 to form pentacycle peptide having formula P7 or a salt thereof and/or stereoisomers thereof and/or isotopic variations thereof:

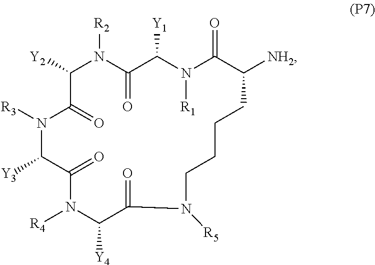

16. The method of claim 15 further comprising removing any protective groups from Y₁, Y₂, Y₃, and Y₄ to form:

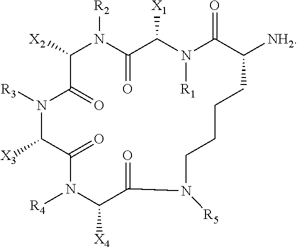

17. The method of claim 15, wherein P₁ is removeable under different conditions that P₂, P₃, P₄, and P₅.

18. The method of claim 15, wherein P₁ is carbobenzyloxy and P₂, P₃, P₄, and P₅ are tert-butoxycarbonyl.

19. The method of claim 15, wherein Y₁ is isopropyl.

20. The method of claim 15, wherein Y₂ is a protected p-hydroxybenzyl.

21. The method of claim 15, wherein Y₃ is methyl.

22. The method of claim 15, wherein Y₄ is benzyl.

23. A method for forming an anabaenopeptin or derivative thereof, the method comprising:
a) reacting the compound of claim 1 with a compound having formula III 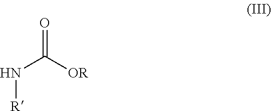
to form the anabaenopeptin or derivative thereof having formula IV: 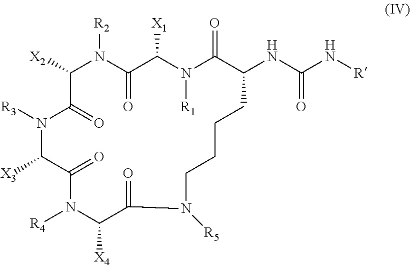
wherein:
R is C₁₋₁₀ alkyl, phenyl, or nitrophenyl; and
R′ is a C₄₋₂₀ hydrocarbon group that includes an ester moiety, a C₄₋₂₀ alkyl, C₆₋₂₀ aryl, or a C₇₋₂₀ alkyl aryl.

24. The method of claim 23 wherein the compound having formula III is selected from the group consisting of:

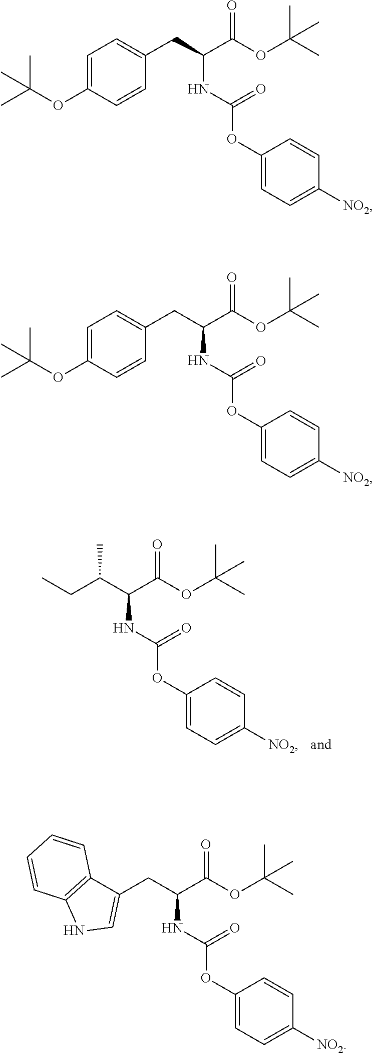 [SMILES: CC(C)(C)OC(=O)[C@H](Cc1ccc(OC(C)(C)C)cc1)NC(=O)Oc1ccc([N+](=O)[O-])cc1 ; CC(C)(C)OC(=O)[C@H](Cc1ccc(OC(C)(C)C)cc1)NC(=O)Oc1ccc([N+](=O)[O-])cc1 ; CC[C@H](C)[C@H](NC(=O)Oc1ccc([N+](=O)[O-])cc1)C(=O)OC(C)(C)C ; CC(C)(C)OC(=O)[C@H](Cc1c[nH]c2ccccc12)NC(=O)Oc1ccc([N+](=O)[O-])cc1]

25. A method for forming an anabaenopeptin or derivative thereof, the method comprising:
a) converting the compound having formula of claim 1 into an isocyanate having formula V: 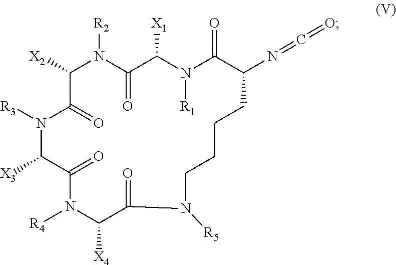
and
b) reacting the compound having formula V with an amine.

26. The method of claim 25 wherein in step a) the compound having formula I is reacted with triphosgene or triphosgene and a base to form the compound having formula V.

27. The method of claim 25 wherein the amine has formula VI: 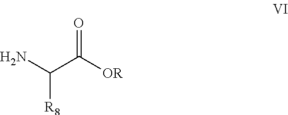
where R₈ is a C₄₋₂₀ hydrocarbon group that includes an ester moiety, a C₄₋₂₀ alkyl, C₆₋₂₀ aryl, or a C₇₋₂₀ alkyl aryl.

28. A method for forming an anabaenopeptin or derivative thereof, the method comprising:
a) reacting the compound of claim 1 with a compound having formula III 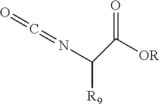
to form the anabaenopeptin or derivative thereof having formula VII: 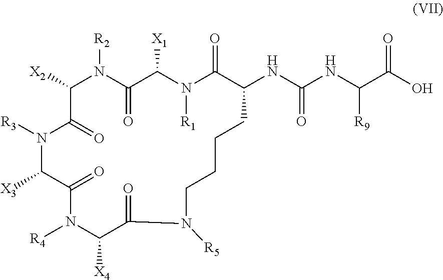
wherein:
R₉ is a C₄₋₂₀ hydrocarbon group, a C₄₋₂₀ alkyl, C₆₋₂₀ aryl, or a C₇₋₂₀ alkyl aryl; and
R′ is a C₄₋₂₀ hydrocarbon group that includes an ester moiety, a C₄₋₂₀ alkyl, C₆₋₂₀ aryl, or a C₇₋₂₀ alkyl aryl.
